(19) United States
(12) Patent Application Publication
(10) Pub. No.: US 2010/0105088 A1
(43) Pub. Date: Apr. 29, 2010

(54) METHOD FOR DIAGNOSING ATHEROSCLEROTIC PLAQUES BY MEASUREMENT OF CD36

(75) Inventors: Aase Handberg, Risskov (DK); Bente Halvorsen, Oslo (NO); Pål Aukrust, Ridabu (NO); Mona Skjelland, Oslo (NO)

Correspondence Address: WEINGARTEN, SCHURGIN, GAGNEBIN & LEBOVICI LLP, TEN POST OFFICE SQUARE, BOSTON, MA, 02109, US

(73) Assignees: AARHUS UNIVERSITET, Arhus C (DK); REGION MIDTJYLLAND, Viborg (DK); RIKSHOSPITALET HF, Oslo (NO)

(21) Appl. No.: 12/525,887

(22) PCT Filed: Feb. 4, 2008
(86) PCT No.: PCT/DK08/00049

§ 371 Date: Aug. 5, 2009

(30) Foreign Application Priority Data
Feb. 5, 2007 (DK) PA 2007 00192

(51) Int. Cl. G01N 33/53; G01N 33/68; C07K 16/00; C07H 21/00

(52) U.S. Cl. 435/7.92; 436/86; 530/389.1; 536/24.3; 536/24.33

(57) ABSTRACT

The present invention relates to diagnosis, classification and monitoring of atherosclerotic plaques in an individual using measurement of the concentration of CD36 in a body fluid and/or the plaque as such. The present invention also relates to diagnosing the burden of atherosclerotic plaques in an individual. Furthermore, the invention relates to a method for diagnosing stenosis caused by atherosclerotic plaques. Within the scope of the present invention are also methods for determining the treatment regime of an individual. Kits and oligonucleotides for use in the methods are claimed.

### TECHNICAL FIELD OF THE INVENTION

The present invention relates to diagnosis, classification and monitoring of atherosclerotic plaques using measurement of the concentration of CD36 in a body fluid and/or the plaque as such.

### BACKGROUND OF THE INVENTION

Atherosclerosis is a progressive inflammatory disease in which lipids, extracellular matrix, macrophages and activated vascular smooth muscle cells accumulate in the arterial wall resulting in growth of an atherosclerotic plaque. Progression of the atherosclerotic lesions can elicit various acute ischemic events such as acute coronary syndromes, transient ischemic attacks (TIA), and stroke due to parts of the plaque being detached and carried with the blood stream until the plaque stops because the size of the plaque exceeds the size of the artery. However, although the participation of inflammatory mediators in the atherosclerotic process has become widely recognized, the identification of the different components, as well as their relative importance, is unclear. In particular, the way in which inflammation may promote the transition from an asymptomatic fibroatheromatous plaque to a vulnerable and symptomatic lesion is not fully understood.

CD36 has numerous cellular functions. It is a Fatty Acid Translocase (FAT) and belongs to the scavenger receptor Class B family, playing a major role in the uptake of Long Chain Fatty Acids (LCFA) over the cell membrane in metabolically active tissue, in foam cell formation, and in uptake of OxLDL by macrophages. The lipid-rich macrophages are then differentiated into or gain the characteristics of foam cells and contribute to the formation of atherosclerotic lesions. In addition, CD36 of macrophages, together with TSP and the integrin alphavbeta3, can phagocytose apoptotic neutrophils. Furthermore, the protein is one of the receptors of collagen in platelet adhesion and aggregation. Moreover, cytoplasmic CD36 plays an important role in signal transduction by interacting with Src family tyrosine kinases. Deficiency in CD36 in Asian and African populations has been associated with insulin resistance.

Alternate names for CD36 (Cluster Determinant 36) include CD36 [HUGO gene name], GPIIIb, GPIV, OKM5-antigen, and PASIV.

### SUMMARY OF THE INVENTION

The present inventors have found that increased concentration of CD36 in a body fluid sample from an individual having an atherosclerotic plaque or in an atherosclerotic plaque as such correlates to an increased risk of said atherosclerotic plaque to become a symptomatic atherosclerotic plaque.

Thus, the present invention relates to methods for diagnosing, classifying and monitoring atherosclerotic plaques, for example in order to distinguish stable atherosclerotic plaques from instable atherosclerotic plaques. In addition it is suggested herein that circulating non-cell bound CD36 protein (soluble CD36, (sCD36)) or a fraction or fragment thereof optionally as part of a lipoprotein complex may be a useful diagnostic or predictive marker for atherosclerotic plaques in blood vessels, in particular in carotid arteries and coronary arteries.

It has been found that the concentration of CD36 both in plasma and in the atherosclerotic plaques increase following the progression of atherosclerotic plaques towards symptomatic atherosclerotic plaques. Accordingly, increase of CD36 concentration may be used as an indication of progression of the atherosclerotic plaques. The method may be used for diagnosing the risk of an individual for acquiring an ischemic event as described herein, and furthermore the method may be used for monitoring an individual for a period observing whether an atherosclerotic plaque is progressing.

Thus, one aspect of the present invention relates to a method for classifying an atherosclerotic plaque(s) in an individual, said method comprising in a sample from said individual, i) determining the concentration of a CD36 polypeptide or part thereof and/or ii) determining the concentration of a CD36 encoding nucleic acid molecule or part thereof, iii) correlating said concentration determined in i) and/or ii) to a standard level, and iv) based on said correlation according to iii) classifying said atherosclerotic plaque.

A second aspect relates to a method for diagnosing an atherosclerotic plaque(s) in an individual, said method comprising in a sample from said individual i) determining the concentration of a CD36 polypeptide or part thereof and/or ii) determining the concentration of a CD36 encoding nucleic acid molecule or part thereof, iii) correlating said concentration determined in i) and/or ii) to a standard level, and iv) based on said correlation according to iii) diagnosing said atherosclerotic plaque(s).

A third aspect of the present invention pertains to a method for monitoring an atherosclerotic plaque(s) in an individual, said method comprising in a sample from said individual i) determining the concentration of a CD36 polypeptide or part thereof and/or ii) determining the concentration of a CD36 encoding nucleic acid molecule or part thereof, iii) correlating said concentration determined in i) and/or ii) to a concentration of a CD36 polypeptide or part thereof and/or the concentration of a CD36 encoding nucleic acid molecule or part thereof, measured in the same individual previously and/or iv) correlating said concentration determined in i) and/or ii) to a standard level, and v) based on said correlation according to iii) and/or iv) monitoring any progress of said atherosclerotic plaques.

A fourth aspect relates to a method for diagnosing an individual at risk of having and/or acquiring a symptomatic atherosclerotic plaque(s), said method comprising in a sample from said individual, i) determining the concentration of a CD36 polypeptide or part thereof and/or ii) determining the concentration of a CD36 encoding nucleic acid molecule or part thereof, iii) correlating said concentration determined in i) and/or ii) to a standard level, and iv) based on said correlation according to iii) diagnosing whether said individual is at risk of having and/or acquiring a symptomatic atherosclerotic plaque.

In a further aspect the present invention relates to a method for diagnosing burden of an atherosclerotic plaques in an individual, said method comprising in a sample from said individual i) determining the concentration of a CD36 polypeptide or part thereof and/or ii) determining the concentration of a CD36 encoding nucleic acid molecule or part thereof, iii) correlating said concentration determined in i) and/or ii) to a standard level, and iv) based on said correlation according to iii) diagnosing the burden of atherosclerotic plaque(s) in said individual.

In yet a further aspect the present invention pertains to a method for diagnosing stenosis caused by atherosclerotic plaques in an individual, said method comprising in a sample from said individual i) determining the concentration of a CD36 polypeptide or part thereof and/or ii) determining the concentration of a CD36 encoding nucleic acid molecule or part thereof, iii) correlating said concentration determined in i) and/or ii) to a standard level, and iv) based on said correlation according to iii) diagnosing the degree of stenosis caused by atherosclerotic plaques in an individual.

Another aspect relates to a method for determining the treatment regime of an individual, said method comprising in a sample from said individual, i) determining the concentration of a CD36 polypeptide or part thereof and/or ii) determining the concentration of a CD36 encoding nucleic acid molecule or part thereof, iii) correlating said concentration determined in i) and/or ii) to a standard level, and iv) based on said correlation according to iii) diagnosing the degree of stenosis caused by atherosclerotic plaques and/or diagnosing the risk of having and/or acquiring a symptomatic atherosclerotic plaque in an individual v) deciding on the treatment regime of said individual based on the outcome of iv).

In a final aspect the present invention relates to a kit for use in the methods as disclosed herein, wherein the kit comprises at least one detection member. It is appreciated that the detection member is at least one antibody, wherein said antibody is directed against CD36 polypeptide or part thereof. Furthermore, it is to be understood that the detection member is selected from the group consisting of at least one primers, at least one probe and at least one primer pair capable of detecting a CD36 encoding nucleic acid molecule.

### DRAWINGS

FIG. 1:

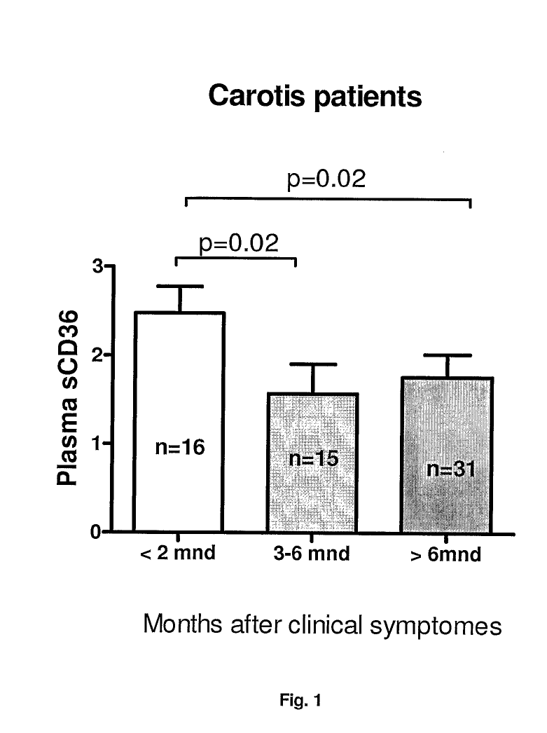

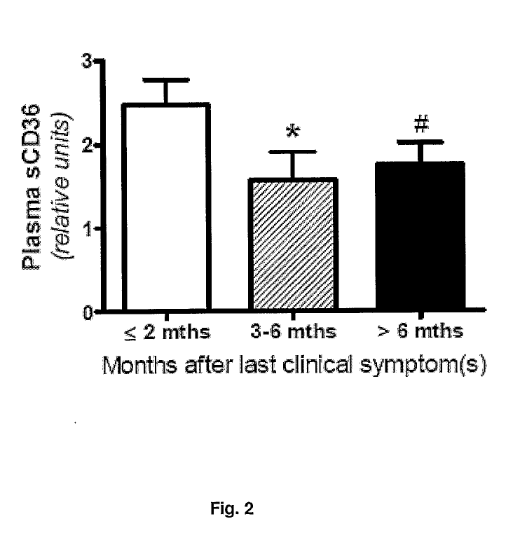

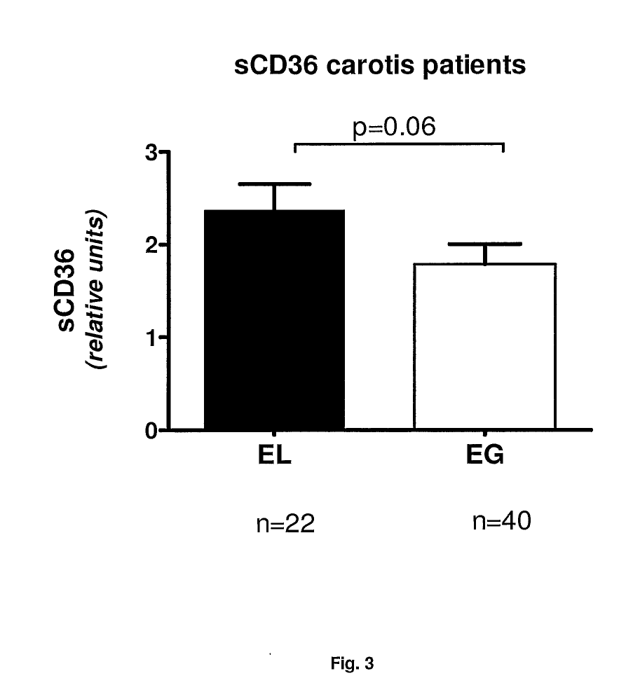

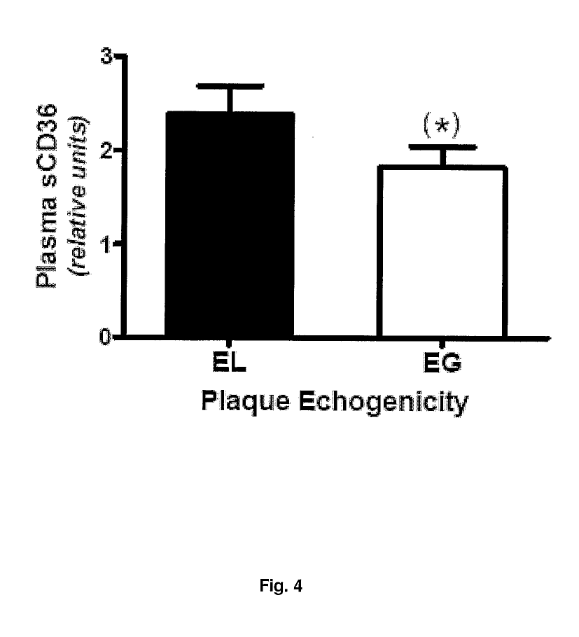

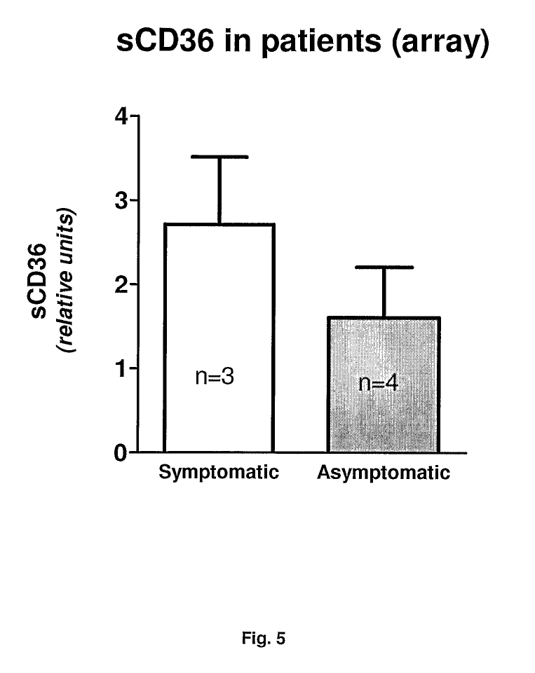

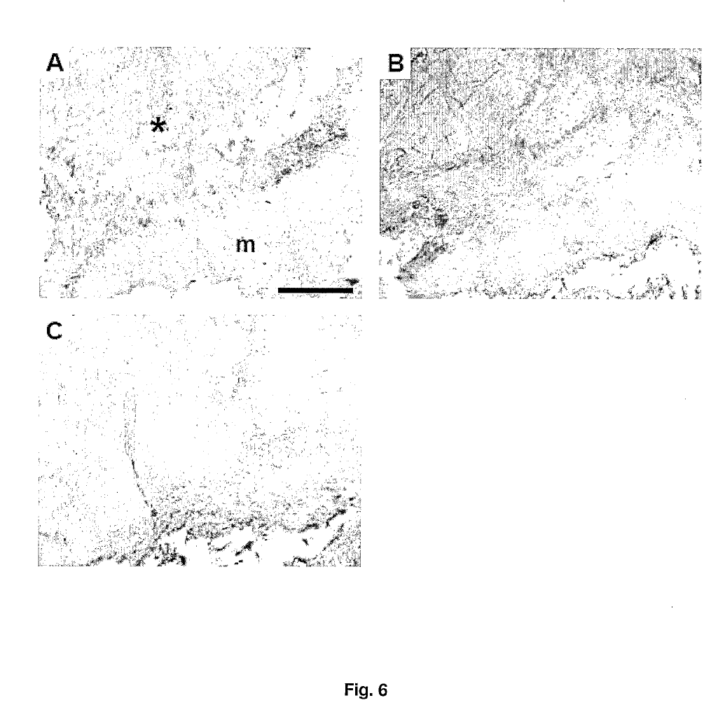

Soluble CD36 (sCD36) in plasma from patients with carotid atherosclerosis according to the plaque stability. Data are given as function of time period since last clinical symptoms. The carotid stenosis was diagnosed and classified by pre-cerebral color Duplex ultrasound (1) and CT angiography (2) according to consensus criteria. sCD36 is higher in patients who had experienced clinical symptoms within the last 2 months (<2 mnd), as compared to patients who experienced symptoms 3-6 months (3-6 mnd) or more than 6 months (>6 mnd), since last symptoms before examination and blood sampling. N=number of patients, p=level of significance (Mann-Whitney U test). Plasma sCD36 is measured as relative units, i.e. to a pool of EDTA plasma from the routine hospital blood sampling. Clinical symptoms of plaques designate the presence of cerebrovascular symptoms. Symptomatic atherosclerotic plaques are plaques in patients with stroke, TIA (transient ischemic attack), or amaurosis fugax ipsilateral to the stenotic internal carotid artery. Error bars represent SEM.
(1) Grant E G, Benson C B, Moneta G L et al. Carotid artery stenosis: gray-scale and Doppler US diagnosis—Society of Radiologists in Ultrasound Consensus Conference, Radiology. 2003; 229:340-346
(2) Anderson G B, Asforth R, Steinke D E et al. CT angiography for the detection and characterization of carotid artery bifurcation disease. Stroke. 2000; 31:2168-2174.

FIG. 2:

Plasma levels of soluble CD36 (sCD36) in patients with atherosclerotic carotid stenosis. The patients were divided into groups according to months (mths) after their last clinical symptoms. Clinical symptoms within the last 2 mths (open bar, n=16), 3-6 mths (squared bar, n=15), or more than 6 mths (black bar, n=31). *, p<0.02 versus 2 mths; #, p<0.02 versus 2 mths.

FIG. 3:

Soluble CD36 (sCD36) in plasma from patients with carotid atherosclerosis. The carotid stenosis was diagnosed and classified by pre-cerebral color Duplex ultrasound (1) and CT angiography (2) according to consensus criteria. The patients were divided into Echolucent (EL) and Echogenic (EG) dependent on ultrasound classification. N=number of patients, p=level of significance (Mann-Whitney U test). Plasma sCD36 is measured as relative units, i.e. to a pool of EDTA plasma from the routine hospital blood sampling. Error bars represent SEM.
(1) Grant E G, Benson C B, Moneta G L et al. Carotid artery stenosis: gray-scale and Doppler US diagnosis—Society of Radiologists in Ultrasound Consensus Conference, Radiology. 2003; 229:340-346
(2) Anderson G B, Asforth R, Steinke D E et al. CT angiography for the detection and characterization of carotid artery bifurcation disease. Stroke. 2000; 31:2168-2174.

FIG. 4:

Plasma levels of soluble CD36 (sCD36) in patients with atherosclerotic carotid stenosis. The patients were grouped based on echogenicity after ultrasound examination of the atherosclerotic carotid artery. EL, echolucent plaques (n=20); EG, echogenic/heterogeneous plaques (n=39). (*), p=0.09.

FIG. 5:

Atherosclerotic plaques from the internal carotid artery from endarterectomy patients were classified into two groups depending on whether or not the patients had experienced ipsilateral stroke, TIA or amaurosis fugax in the prior six months to surgery. Plaques were characterized as symptomatic (n=3) or asymptomatic (n=4) according to the presence or absence of cerebrovascular symptoms, respectively. Soluble CD36 (sCD36) was measured in plasma from blood samples drawn the day prior to surgery. The patients were identical to the patients included in the gene expression profile analysis (microarrays) of carotid plaque (Table 1) with the exception that the blood sample from one of the patients was missing and therefore sCD36 was not measured. N=number of patients. Plasma sCD36 is measured as relative units, i.e. to a pool of EDTA plasma from the routine hospital blood sampling. Error bars represent SEM.

FIG. 6:

Representative photomicrographs of serial sections demonstrating anti-CD36 (A), anti-calprotectin (B), and anti-smooth muscle actin (C) immunostaining in an atherosclerotic carotid lesion removed by endarterectomy from a patient with unstable disease. CD36 immunoreactivity was localized to parts of the lipid-rich core (*) of the lesion with numerous CD68-positive macrophages as seen in panel B. No immunostaining was seen outside the core. Strongest anti-CD36 immunostaining was seen in regions adjacent to the media (m). Panel C demonstrates anti-smooth muscle actin immunostaining in the media. Scale bar 100 μm.

### DETAILED DESCRIPTION OF THE INVENTION

### Definitions

Atherosclerotic plaques: the term is used in it's conventional meaning, ie. accumulation in the arterial wall of lipids, extracellular material, inflammatory mediators, inflammatory cells (i.e. leukocytes), macrophages and vascular smooth muscle cells.

Symptomatic atherosclerotic plaque: an atherosclerotic plaque in an individual that has suffered from symptoms, for example in the form of ipsilateral stroke, transient ischemic attack, or amaurosis fugax.

Asymptomatic atherosclerotic plaque: an atherosclerotic plaque in an individual that has not experienced the symptoms mentioned under symptomatic atherosclerotic plaque within the past six months or more.

Stable atherosclerotic plaque: an atherosclerotic plaque in an individual that shows no sign of progression during a predefined time-period of six months or more.

Instable atherosclerotic plaque: an atherosclerotic plaque in an individual show signs of progression.

In the present context the term atheromatous plaque is the accumulation and swelling in artery walls where the swelling is caused by for example macrophages, cell debris, lipids such as cholesterol and fatty acids, calcium and fibrous connective tissue. Thus, the process of atheroma development within an individual is called atherogenesis and the overall result of the disease process is termed atherosclerosis. Therefore, the term atheromatous plaque is used interchangeably with atherosclerotic plaque in the present invention.

By the term ‘CD36 encoding nucleic acid molecule’ is meant a transcriptional product of the gene encoding CD36.

Diagnosis: as used herein refers to methods by which the skilled person can estimate and/or determine whether or not a patient is suffering from a given disease or condition. The skilled person often makes a diagnosis on the basis of one or more diagnostic indicators, i.e., in the amount or concentration of a marker, or change in amount of marker which is indicative of the presence, severity, or absence of the condition.

The term correlating as used herein, refers to comparing the presence or amount of the marker(s) in a patient to its presence or amount in individuals suffering from or at risk of suffering from, a given condition; or in persons known to be free of a given condition. As discussed above, a marker level in a patient sample can be compared to a level known to be associated with a specific diagnosis. The sample's marker level is said to have been correlated with a diagnosis; that is, the skilled person can use the marker level to determine whether the patient suffers from a specific type diagnosis, and respond accordingly. Alternatively, the sample's marker level can be compared to a marker level known to be associated with a good outcome (e.g., the absence of disease, etc.).

It is appreciated that the term ‘marker’ in the present invention refers to CD36 polypeptide and/or CD36 encoding nucleic acid molecule.

Determining the diagnosis as used herein refers to methods by which the skilled person can determine the presence or absence of a particular disease in a patient. The term “diagnosis” does not refer to the ability to determine the presence or absence of a particular disease with 100% accuracy. Instead, the skilled person will understand that the term “diagnosis” refers to an increased probability that a certain disease is present in the subject. In preferred embodiments, a diagnosis indicates about a 5% increased chance that a disease is present, about a 10% chance, about a 15% chance, about a 20% chance, about a 25% chance, about a 30% chance, about a 40% chance, about a 50% chance, about a 60% chance, about a 75% chance, about a 90% chance, and about a 95% chance. The term “about” in this context refers to +/−2%.

### Diagnosis, Classification and Monitoring Atherosclerotic Plaque Status

The present invention is based on the analysis of plaques and blood samples from patients who has suffered or suffers from internal carotid stenoses and were treated by carotid endarterectomy or carotid angioplasty with stenting. Based on the outcome of a study of the concentration of CD36 polypeptide and/or CD36 encoding nucleic acid molecule in samples from patients the present invention allows for monitoring progression toward an symptomatic plaque.

The destabilization of an atherosclerotic plaque leading to the progression of an atherosclerotic plaque from asymptomatic to symptomatic is important to diagnose, classify, and monitor since the destabilization can elicit various acute ischemic events, such as stroke, transient ischemic attack, and acute coronary syndromes.

The present invention relates to a method for diagnosing, classifying and monitoring atherosclerotic plaques in an individual in order to predict and prevent acute ischemic events due to destabilization of the plaques.

The method may be used for diagnosing, classifying and monitoring atherosclerotic plaques in any artery, in particular in the carotid arteries and the coronary arteries as well as a method for determining whether an individual is at risk of having and/or acquiring a symptomatic atherosclerotic plaque.

Furthermore, the present invention relates to a method for determining the atherosclerotic plaque burden, ie. the amount of plaques in the individual, by measuring the concentration of CD36 polypeptide or CD36 nucleic acid molecule.

Also, the present invention relates to a method for determining the degree of stenosis caused by an atherosclerotic plaque, by measuring the concentration of CD36 polypeptide or CD36 nucleic acid molecule.

Furthermore, the present invention relates to a method for determining the treatment regime of an individual, by measuring the concentration of CD36 polypeptide or CD36 nucleic acid molecule.

Thus, the present invention relates in one aspect to a method for classifying atherosclerotic plaques in an individual, said method comprising in a sample from said individual, i) determining the concentration of a CD36 polypeptide or part thereof and/or ii) determining the concentration of a CD36 encoding nucleic acid molecule or part thereof, iii) correlating said concentration determined in i) and/or ii) to a standard level, and iv) based on said correlation according to iii) classifying said atherosclerotic plaque.

In another aspect the invention relates to a method for diagnosing atherosclerotic plaques in an individual, said method comprising in a sample from said individual i) determining the concentration of a CD36 polypeptide or part thereof and/or ii) determining the concentration of a CD36 encoding nucleic acid molecule or part thereof, iii) correlating said concentration determined in i) and/or ii) to a standard level, and iv) based on said correlation according to iii) diagnosing said atherosclerotic plaques.

The classification and/or diagnosis of atherosclerotic plaques involves resolving whether an asymptomatic plaques is destabilized and has a higher risk of progressing into symptomatic plaques which may result in the development of acute ischemic events. In the present invention a correlation has been found between the concentration of CD36 polypeptide and/or CD36 encoding nucleic acid molecule and the destabilization of asymptomatic plaques to symptomatic plaques. Thus, an increase in the concentration of CD36 polypeptide and/or CD36 encoding nucleic acid compared to a standard level is indicative of destabilization of asymptomatic plaques progressing into symptomatic plaques.

In yet another aspect the invention relates to a method for monitoring an atherosclerotic plaque(s) in an individual, said method comprising in a sample from said individual i) determining the concentration of a CD36 polypeptide or part thereof and/or ii) determining the concentration of a CD36 encoding nucleic acid molecule or part thereof, iii) correlating said concentration determined in i) and/or ii) to a concentration of a CD36 polypeptide or part thereof and/or the concentration of a CD36 encoding nucleic acid molecule or part thereof, measured in the same individual previously and/or iv) correlating said concentration determined in i) and/or ii) to a standard level, and v) based on said correlation according to iii) and/or iv) monitoring any progress of said atherosclerotic plaques.

It is thus within the scope of the present invention to monitor the progression of destabilization of atherosclerotic plaques, whether asymptomatic or symptomatic in an individual. The concentration of CD36 polypeptide and/or CD36 encoding nucleic acid molecule is determined as described in steps i) and/or ii) and correlated to a standard level. The outcome of the correlation can be compared to concentrations of CD36 polypeptide and/or CD36 encoding nucleic acid molecule determined at other time points in said individual. By monitoring the concentration of CD36 polypeptide and/or CD36 encoding nucleic acid molecule in an individual at different time points the progress of the destabilization of atherosclerotic plaques can be followed. Such monitoring of an individual can be useful for determining the treatment regime of an individual as described elsewhere herein.

In a further aspect the invention relates to a method for diagnosing an individual at risk of having and/or acquiring a symptomatic atherosclerotic plaques, said method comprising in a sample from said individual, i) determining the concentration of a CD36 polypeptide or part thereof and/or ii) determining the concentration of a CD36 encoding nucleic acid molecule or part thereof, iii) correlating said concentration determined in i) and/or ii) to a standard level, and iv) based on said correlation according to iii) diagnosing whether said individual is at risk of having and/or acquiring a symptomatic atherosclerotic plaque.

In yet a further aspect the invention relates to a method for diagnosing burden of atherosclerotic plaques in an individual, said method comprising in a sample from said individual i) determining the concentration of a CD36 polypeptide or part thereof and/or ii) determining the concentration of a CD36 encoding nucleic acid molecule or part thereof, iii) correlating said concentration determined in i) and/or ii) to a standard level, and iv) based on said correlation according to iii) diagnosing the burden of atherosclerotic plaques in said individual.

Furthermore, the present invention relates to a method for diagnosing stenosis caused by atherosclerotic plaques in an individual, said method comprising in a sample from said individual i) determining the concentration of a CD36 polypeptide or part thereof and/or ii) determining the concentration of a CD36 encoding nucleic acid molecule or part thereof, iii) correlating said concentration determined in i) and/or ii) to a standard level, and iv) based on said correlation according to iii) diagnosing the degree of stenosis caused by atherosclerotic plaques in an individual.

In addition, the present invention relates to a method for determining the treatment regime of an individual, said method comprising in a sample from said individual, i) determining the concentration of a CD36 polypeptide or part thereof and/or ii) determining the concentration of a CD36 encoding nucleic acid molecule or part thereof, iii) correlating said concentration determined in i) and/or ii) to a standard level, and iv) based on said correlation according to iii) diagnosing the degree of stenosis caused by atherosclerotic plaques and/or diagnosing the risk of having and/or acquiring a symptomatic atherosclerotic plaque in an individual v) deciding on the treatment regime of said individual based on the outcome of iv). It is appreciated that the effect of the treatment can be monitored subsequently.

The present invention relates to atherosclerotic plaques in all types of arteries of the body, for example carotids, coronary arteries, renal artery or arteries supplying the arms and legs. Below are listed some of the symptoms that are associated with atherosclerosis and the destabilization of atherosclerotic plaques and an increased risk of-progression into symptomatic atherosclerotic plaques.

A stenosis is an abnormal narrowing in a blood vessel. Stenosis of the vascular type are often associated with a noise (bruit) resulting from turbulent flow over the narrowed blood vessel. This bruit can be made audible by a stethoscope. However, other more precise methods of diagnosis includes ultrasound, magnetic resonance imaging/magnetic resonance angiography, computed tomography/CT-Angiography which display anatomic imaging and/or flow phenomena.

Within the scope of the present invention are also methods related to determination, classification, diagnosing, monitoring coronary heart disease (CHD), which is also known as coronary artery disease (CAD), ischemic heart disease, atherosclerotic heart disease. Coronary heart disease is the end result of the accumulation of atheromatous plaques within the walls of the arteries that supply the myocardium with oxygen and nutrients.

Typically, the symptoms of coronary heart disease are noted only in the advanced state of disease. The majority of individuals suffering from coronary heart disease do not show any evidence of disease for years and even decades while the disease progresses until the first onset of symptoms, often a “sudden” heart attack, arises. The reason for this disease progression is often rupture of atheromatous plaques may rupture and start limiting blood flow to the heart muscle.

Acute myocardial infarction which is also commonly known as a heart attack is also a condition to which the present invention relates. Acute myocardial infarction is a condition which involves the interruption of the blood supply to part of the heart. The interruption of the blood supply is often brought on by the rupture of an atherosclerotic plaque of the inner arterial wall. The atherosclerotic plaque may block the passage of blood in the artery which results in ischemia or oxygen shortage which causes damage and potential death of heart tissue.

Individuals at risk of having and/or acquiring an acute myocardial infarction are normally characterized by the use of risk factors, such as a previous history of vascular disease for example atherosclerotic coronary heart disease and/or angina, a previous heart attack or stroke.

Angina pectoris is mainly caused by coronary artery disease is chest pain due to lack of blood and hence oxygen supply of the heart muscle which is generally due to obstruction or spasm of the coronary arteries. Thus, according to one embodiment of the invention the methods and kits herein are also related to determine whether an individual is at risk of having/acquiring angina pectoris caused by atherosclerotic plaque.

Carotid stenosis is a narrowing of the lumen of the carotid artery, usually caused by atherosclerosis. The unqualified term “carotid stenosis” in common medical usage refers to the stenosis in the proximal part of the internal carotid artery (at the carotid bulb), as this is the by far the most common site of stenosis within the carotid arteries. Stenosis in other parts of the carotid arteries does occur. Thus, in the present context the term ‘carotid stenosis’ covers the narrowing in at least one of the carotid arteries, and in particular the proximal part of the internal carotid artery.

Atherosclerotic carotid stenosis may be asymptomatic or it may cause symptoms by embolism to either cerebral vessels in the brain or to the retinal arteries. The term emboli as used herein is used in its conventional meaning, namely the event in which an embolus (object) through circulation migrates from one part of the body and causes a blockage of a blood vessel in another part of the body. Emboli to the cerebral arteries cause transient ischaemic attack (TIA) or cerebrovascular accident (CVA). Emboli to the retina produce amaurosis fugax or retinal infarction.

The diagnosis of carotid stenosis is often determined by colour flow duplex ultrasound scan of the neck arteries. This test has moderate sensitivity and specificity and yields many false-positive results.

The methods and kits of the present invention also relate to a transient ischemic attack, TIA, which is often referred to as a “mini stroke”. TIA is caused by the temporary disturbance of blood supply to a restricted area of the brain. The disturbance of the blood supply to the brain often results in brief neurologic dysfunction that usually persists for less than 24 hours. The symptoms associated with TIA vary widely depending on the area of the brain affected by the TIA. Frequently encountered symptoms include temporary loss of vision (typically amaurosis fugax); difficulty speaking (aphasia); weakness on one side of the body (hemiparesis); and numbness or tingling (paresthesia), usually on one side of the body. Impairment of consciousness is very uncommon. If there are neurological symptoms persisting for more than 24 hours, it is classified as a cerebrovascular accident, or stroke.

The clinical feature of stroke or cerebrovascular accident (CVA) is a rapidly developing loss of brain function due to a disturbance in the blood supply to the brain. This disturbance may be due to lack of blood supply (ischemia) caused by thrombosis or embolism, or due to a hemorrhage. The traditional definition of stroke is of a “neurological deficit of cerebrovascular cause that persists beyond 24 hours or is interrupted by death within 24 hours”. In the present invention the methods and kits are related to stroke caused by atherosclerotic plaques which cause disturbance in the blood supply to the brain.

Previous stroke or transient ischemic attack are typical risk factors of stroke.

### CD36

For the purposes of the present invention the term “CD36” used in phrases, such as “circulating CD36”, or “non-cell bound CD36”, and in the claims, includes CD36 protein or a fragment thereof which is recognised by a specific antibody against CD36, such as sc5522, sc9154, and sc7309. This includes full length CD36 protein, a polypeptide or peptide fragment thereof, as well as such protein or fragment(s) thereof present in a high molecular weight plasma fraction complex comprising lipoprotein thereof all of which are preferably soluble (sCD36). The terms circulating CD36 and soluble CD36 (sCD36) may be used as synonyms herein. The circulating CD36 may be secreted from caveolae or which is part of a microparticle comprising membrane portions originating from a caveola membrane which has been released from a cell membrane and is present in the blood circulation. Caveolae of interest in this connection may be present in the cell membrane of cells, such as adipocytes, macrophages, monocytes and thrombocytes.

CD36 is a 471 amino acid, transmembrane protein (having 1 or 2 membrane spanning domains at amino acid residue positions 439-460 and possibly 7-28). CD36 is a highly glycosylated, 88 kDa glycoprotein with palmitoyl binding sites. CD36 is present in caveolae where it may play a role in the mediation of cellular cholesterol movement into and out of cells.

Molecular families in which CD36 is a member:

CD36->SR-B class->host defence scavenger receptors->scavenger receptor super-family

Molecular structure of CD36:

471 amino acid residues;
Transmembrane region (residues 439-465) and,
438 amino acid amino-terminal region may be entirely extracellular or may have a second potential transmembrane region near the amino terminal end;
aa short cytoplasmic tail (residues 466-471);
Within the extracellular region resides a hydrophobic region which probably associ-ates with the outer cell membrane (residues 184-204).

The molecular mass of CD36 is reported to be dependent on cell type as shown below:

Platelets 88 kDa/113 kDa

### Fetal Erythrocytes 78 kDa

Mammary epithelial Cells 85 kDa
Erythroleukeimic 88 kDa, 85 kDa, 57 kDa8

### HeLa 85 (160) kDa 85 kDa

Dermal Microvascular endothelial cells 80-90 kDa

In post-transcriptional modification of CD36 two alternate CD36 mRNA forms have been identified. The first mRNA type is expressed in HEL cells and omits amino acid residues 41-143. The second mRNA type has not yet been translated but in which the last 89 residues have been omitted. Thus, CD36 is also found in forms in which the open reading frame is 90-1708 and 357-1775, respectively.

Post-translational modification of CD36:

CD36 is purported to be heavily glycosylated, with 10 N-linked glycosylation sites in the extracellular portion. Glycosylation has been suggested to confer its resistance to proteolytic cleavage;
Threonine 92 has been shown to be phosphorylated;
CD36 is also palmitoylated on both N- and C-terminal cytoplasmic tails.

Proteins and DNA elements which regulate transcription of CD36 molecule:

Oct-2: The first gene shown to be regulated by the Oct-2 transcription factor during B cell differentiation;
PEBP2: The PEBP2/CBF transcription factors may be important for the constitutive expression of CD36 in monocyte;
CBF: The PEBP2/CBF transcription factors may be important for the constitutive expression of CD36 in monocyte.

Substrates for CD36 are unknown. It may be possible that CD36 regulates autophosphorylation of residue Thr92. Enzymes which modify CD36 are unknown. It may be possible that Thr92 is phosphorylated by extracellular threonine kinase(s). Intracellular signalling is probably associated with phosphorylation of Fyn, Lyn and Yes, but the manner by which the cytoplasmic tail interacts with these PTKs is unknown.

Main cellular expression of CD36:

CD36 is expressed on platelets, mature monocytes and macrophages, microvascular endo-thelial cells, mammary endothelial cells, during stages of erythroid cell development and on some macrophage derived dendritic cells, muscle cells, liver cells, and adipocytes.

The physiological events regulated by CD36 ligation are still very much unknown. Up to 50% of oxidized LDL are ingested through CD36, thus CD36 appears to be a major scavenger receptor. However, given the apparent absence of disease states in CD36 deficient subjects, other mechanisms appear to be capable of compensating for its absence.

### CD36 Sequences

The CD36 polypeptide and/or CD36 nucleic acid molecule assessed by the method according preferably have one of the sequences shown below. Furthermore, a nucleic acid molecule may be a RNA nucleic acid molecule being complementary to one or more of the sequences shown below:

### Gene Symbol CD36 HGNC

### Chromosomal Location 7q11.2

### UniGene ID

### Build 191 (7 May 2006)

### Hs. 120949 NCBI (FULL LENGTH)

### Hs. 248425

### Hs. 75613

### Hs. 325823

Entrez Gene 948 Entrez gene

### HGNC: 1663

### HPRD: 01430

### SwissProt

### P16671 EMBL-EBI

### OMIM

### 173510 NCBI

### 248310 NCBI

### 608404 NCBI

### Reference Sequences

### Protein ID

### NP_{—}000063.2 NCBI

### NP_{—}001001547.1 NCBI

### NP_{—}001001548.1 NCBI

### Transcript ID

### NM_{—}000072 NCBI

### NM_{—}001001547 NCBI

### NM_{—}001001548 NCBI

### Nucleotide Sequence:

| (SEQ ID NO.: 1) | |
|---|---|
| NM_001001548 2338 bp mRNA linear PRI 20-JAN-2008 | |
| Homo sapiens CD36 molecule (thrombospondin receptor) (CD36), | |
| transcript variant 1, mRNA. | |
| ACCESSION NM_001001548 (VERSION NM_001001548.1) | |
| ORIGIN | |
| 1 | gaggactgca gtgtaggact ttcctgcaga ataccatttg atcctattaa gaattgtcca |
| 61 | aatgttggag catttgattg aaaaatcctt cttagccatt ttaaagatag ctttccaatg |
| 121 | attagacgaa ttgattcttt ctgtgactca tcagttcatt tcctgtaaaa ttcatgtctt |
| 181 | gctgttgatt tgtgaataag aaccagagct tgtagaaacc actttaatca tatccaggag |
| 241 | tttgcaagaa acaggtgctt aacactaatt cacctcctga acaagaaaaa tgggctgtga |
| 301 | ccggaactgt gggctcatcg ctggggctgt cattggtgct gtcctggctg tgtttggagg |
| 361 | tattctaatg ccagttggag acctgcttat ccagaagaca attaaaaagc aagttgtcct |
| 421 | cgaagaaggt acaattgctt ttaaaaattg ggttaaaaca ggcacagaag tttacagaca |
| 481 | gttttggatc tttgatgtgc aaaatccaca ggaagtgatg atgaacagca gcaacattca |
| 541 | agttaagcaa agaggtcctt atacgtacag agttcgtttt ctagccaagg aaaatgtaac |
| 601 | ccaggacgct gaggacaaca cagtctcttt cctgcagccc aatggtgcca tcttcgaacc |
| 661 | ttcactatca gttggaacag aggctgacaa cttcacagtt ctcaatctgg ctgtggcagc |
| 721 | tgcatcccat atctatcaaa atcaatttgt tcaaatgatc ctcaattcac ttattaacaa |
| 781 | gtcaaaatct tctatgttcc aagtcagaac tttgagagaa ctgttatggg gctataggga |
| 841 | tccatttttg agtttggttc cgtaccctgt tactaccaca gttggtctgt tttatcctta |
| 901 | caacaatact gcagatggag tttataaagt tttcaatgga aaagataaca taagtaaagt |
| 961 | tgccataatc gacacatata aaggtaaaag gaatctgtcc tattgggaaa gtcactgcga |
| 1021 | catgattaat ggtacagatg cagcctcatt tccacctttt gttgagaaaa gccaggtatt |
| 1081 | gcagttcttt tcttctgata tttgcaggtc aatctatgct gtatttgaat ccgacgttaa |
| 1141 | tctgaaagga atccctgtgt atagatttgt tcttccatcc aaggcctttg cctctccagt |
| 1201 | tgaaaaccca gacaactatt gtttctgcac agaaaaaatt atctcaaaaa attgtacatc |
| 1261 | atatggtgtg ctagacatca gcaaatgcaa agaagggaga cctgtgtaca tttcacttcc |
| 1321 | tcattttctg tatgcaagtc ctgatgtttc agaacctatt gatggattaa acccaaatga |
| 1381 | agaagaacat aggacatact tggatattga acctataact ggattcactt tacaatttgc |
| 1441 | aaaacggctg caggtcaacc tattggtcaa gccatcagaa aaaattcaag tattaaagaa |
| 1501 | tctgaagagg aactatattg tgcctattct ttggcttaat gagactggga ccattggtga |
| 1561 | tgagaaggca aacatgttca gaagtcaagt aactggaaaa ataaacctcc ttggcctgat |
| 1621 | agaaatgatc ttactcagtg ttggtgtggt gatgtttgtt gcttttatga tttcatattg |
| 1681 | tgcatgcaga tcgaaaacaa taaaataaac ctggctcaag cacaaaccaa tttgtgttgt |
| 1741 | tctgattcaa taattggttt ctgggtggcc aattcagaag aagagtgtac atgctcaaca |
| 1801 | aatcctaggc cctgcattcc tgtcatcctc atccggggga aacaccatca tcccagtagc |
| 1861 | tgccctattc aactgcaaca gtctccagga ccatcagtat actgcatttc atgtgcacca |
| 1921 | aatattttga aagacattta taaataattg gcttatgact catatttctc tatgaatacc |
| 1981 | ttcatacagc aggtataact cttttcttta tgggcttaaa tattttgtca ctgatcctgc |
| 2041 | aaatggacat cattttagca cactagcggt ttatatttta aggaccttca ttctctgttc |
| 2101 | tgcacctctt ctggaaattg agtaaatttt gctttttttt ttttactcag ttgcaactta |
| 2161 | cgcttggcat cttcagaatg cttttctagc attaagagat gtaaatgata aaggaattat |
| 2221 | tgtatgaaat attacaaagc gtagactatg cattgttatt cattataata ttttttgctg |
| 2281 | tcataatcgc ctcataaaga caggtttcaa ccattaaaat atgttcttcc ttaaaaaa |
| // | |
| ACCESSION NM_000072 (VERSION NM_000072.2) | |
| (SEQ ID NO.: 2) | |
| NM_000072 1983 bp mRNA linear PRI 20-JAN-2008 | |
| DEFINITION Homo sapiens CD36 molecule (thrombospondin receptor) | |
| (CD36), transcript variant 3, mRNA. | |
| ORIGIN | |
| 1 | gaggactgca gtgtaggact ttcctgcaga ataccatttg atcctattaa gaattgtcca |
| 61 | aatgttggag catttgattg aaaaatcctt cttagccatt ttaaagatag ctttccaatg |
| 121 | attagacgaa ttgattcttt ctgtgactca tcagttcatt tcctgtaaaa ttcatgtctt |
| 181 | gctgttgatt tgtgaataag aaccagagct tgtagaaacc actttaatca tatccaggag |
| 241 | tttgcaagaa acaggtgctt aacactaatt cacctcctga acaagaaaaa tgggctgtga |
| 301 | ccggaactgt gggctcatcg ctggggctgt cattggtgct gtcctggctg tgtttggagg |
| 361 | tattctaatg ccagttggag acctgcttat ccagaagaca attaaaaagc aagttgtcct |
| 421 | cgaagaaggt acaattgctt ttaaaaattg ggttaaaaca ggcacagaag tttacagaca |
| 481 | gttttggatc tttgatgtgc aaaatccaca ggaagtgatg atgaacagca gcaacattca |
| 541 | agttaagcaa agaggtcctt atacgtacag agttcgtttt ctagccaagg aaaatgtaac |
| 601 | ccaggacgct gaggacaaca cagtctcttt cctgcagccc aatggtgcca tcttcgaacc |
| 661 | ttcactatca gttggaacag aggctgacaa cttcacagtt ctcaatctgg ctgtggcagc |
| 721 | tgcatcccat atctatcaaa atcaatttgt tcaaatgatc ctcaattcac ttattaacaa |
| 781 | gtcaaaatct tctatgttcc aagtcagaac tttgagagaa ctgttatggg gctataggga |
| 841 | tccatttttg agtttggttc cgtaccctgt tactaccaca gttggtctgt tttatcctta |
| 901 | caacaatact gcagatggag tttataaagt tttcaatgga aaagataaca taagtaaagt |
| 961 | tgccataatc gacacatata aaggtaaaag gaatctgtcc tattgggaaa gtcactgcga |
| 1021 | catgattaat ggtacagatg cagcctcatt tccacctttt gttgagaaaa gccaggtatt |
| 1081 | gcagttcttt tcttctgata tttgcaggtc aatctatgct gtatttgaat ccgacgttaa |
| 1141 | tctgaaagga atccctgtgt atagatttgt tcttccatcc aaggcctttg cctctccagt |
| 1201 | tgaaaaccca gacaactatt gtttctgcac agaaaaaatt atctcaaaaa attgtacatc |
| 1261 | atatggtgtg ctagacatca gcaaatgcaa agaagggaga cctgtgtaca tttcacttcc |
| 1321 | tcattttctg tatgcaagtc ctgatgtttc agaacctatt gatggattaa acccaaatga |
| 1381 | agaagaacat aggacatact tggatattga acctataact ggattcactt tacaatttgc |
| 1441 | aaaacggctg caggtcaacc tattggtcaa gccatcagaa aaaattcaag tattaaagaa |
| 1501 | tctgaagagg aactatattg tgcctattct ttggcttaat gagactggga ccattggtga |
| 1561 | tgagaaggca aacatgttca gaagtcaagt aactggaaaa ataaacctcc ttggcctgat |
| 1621 | agaaatgatc ttactcagtg ttggtgtggt gatgtttgtt gcttttatga tttcatattg |
| 1681 | tgcatgcaga tcgaaaacaa taaaataagt aagtatgtac caaaaaatat tgcttcaata |
| 1741 | atattagctt atatattact tgttttcact ttatcaaaga gaagttacat attaggccat |
| 1801 | atatatttct agacatgtct agccactgat catttttaaa tataggtaaa taaacctata |
| 1861 | aatattatca cgcagatcac taaagtatat ctttaattct gggagaaatg agataaaaga |
| 1921 | tgtacttgtg accattgtaa caatagcaca aataaagcac ttgtgccaaa gttgtccaaa |
| 1981 | aaa |
| ACCESSION NM_001001547 (VERSION NM_001001547.1) | |
| (SEQ ID NO.: 3) | |
| NM_001001547 2050 bp mRNA linear PRI 20-JAN-2008 | |
| Homo sapiens CD36 molecule (thrombospondin receptor) (CD36), | |
| transcript variant 2, mRNA. | |
| ORIGIN | |
| 1 | agatgtcagg ataaccttaa ggatagatga agggttgaga gcctgtgcct catttctgag |
| 61 | ttctcagctg ctatgccgtg gaaatcctgt ttactttctg catctgctcc tgcaagactc |
| 121 | tggagccagt cttgaggtcc tacatctccg aaagcaagct cttctagaag ttgatagctt |
| 181 | tccaatgatt agacgaattg attctttctg tgactcatca gttcatttcc tgtaaaattc |
| 241 | atgtcttgct gttgatttgt gaataagaac cagagcttgt agaaaccact ttaatcatat |
| 301 | ccaggagttt gcaagaaaca ggtgcttaac actaattcac ctcctgaaca agaaaaatgg |
| 361 | gctgtgaccg gaactgtggg ctcatcgctg gggctgtcat tggtgctgtc ctggctgtgt |
| 421 | ttggaggtat tctaatgcca gttggagacc tgcttatcca gaagacaatt aaaaagcaag |
| 481 | ttgtcctcga agaaggtaca attgctttta aaaattgggt taaaacaggc acagaagttt |
| 541 | acagacagtt ttggatcttt gatgtgcaaa atccacagga agtgatgatg aacagcagca |
| 601 | acattcaagt taagcaaaga ggtccttata cgtacagagt tcgttttcta gccaaggaaa |
| 661 | atgtaaccca ggacgctgag gacaacacag tctctttcct gcagcccaat ggtgccatct |
| 721 | tcgaaccttc actatcagtt ggaacagagg ctgacaactt cacagttctc aatctggctg |
| 781 | tggcagctgc atcccatatc tatcaaaatc aatttgttca aatgatcctc aattcactta |
| 841 | ttaacaagtc aaaatcttct atgttccaag tcagaacttt gagagaactg ttatggggct |
| 901 | atagggatcc atttttgagt ttggttccgt accctgttac taccacagtt ggtctgtttt |
| 961 | atccttacaa caatactgca gatggagttt ataaagtttt caatggaaaa gataacataa |
| 1021 | gtaaagttgc cataatcgac acatataaag gtaaaaggaa tctgtcctat tgggaaagtc |
| 1081 | actgcgacat gattaatggt acagatgcag cctcatttcc accttttgtt gagaaaagcc |
| 1141 | aggtattgca gttcttttct tctgatattt gcaggtcaat ctatgctgta tttgaatccg |
| 1201 | acgttaatct gaaaggaatc cctgtgtata gatttgttct tccatccaag gcctttgcct |
| 1261 | ctccagttga aaacccagac aactattgtt tctgcacaga aaaaattatc tcaaaaaatt |
| 1321 | gtacatcata tggtgtgcta gacatcagca aatgcaaaga agggagacct gtgtacattt |
| 1381 | cacttcctca ttttctgtat gcaagtcctg atgtttcaga acctattgat ggattaaacc |
| 1441 | caaatgaaga agaacatagg acatacttgg atattgaacc tataactgga ttcactttac |
| 1501 | aatttgcaaa acggctgcag gtcaacctat tggtcaagcc atcagaaaaa attcaagtat |
| 1561 | taaagaatct gaagaggaac tatattgtgc ctattctttg gcttaatgag actgggacca |
| 1621 | ttggtgatga gaaggcaaac atgttcagaa gtcaagtaac tggaaaaata aacctccttg |
| 1681 | gcctgataga aatgatctta ctcagtgttg gtgtggtgat gtttgttgct tttatgattt |
| 1741 | catattgtgc atgcagatcg aaaacaataa aataagtaag tatgtaccaa aaaatattgc |
| 1801 | ttcaataata ttagcttata tattacttgt tttcacttta tcaaagagaa gttacatatt |
| 1861 | aggccatata tatttctaga catgtctagc cactgatcat ttttaaatat aggtaaataa |
| 1921 | acctataaat attatcacgc agatcactaa agtatatctt taattctggg agaaatgaga |
| 1981 | taaaagatgt acttgtgacc attgtaacaa tagcacaaat aaagcacttg tgccaaagtt |
| 2041 | gtccaaaaaa |

//

### Protein Sequence:

| (SEQ ID NO.: 4) | |
|---|---|
| ACCESSION NP_001001548(VERSION NP_001001548.1) | |
| ORIGIN | |
| 1 | mgcdrncgli agavigavla vfggilmpvg dlliqktikk qvvleegtia fknwvktgte |
| 61 | vyrqfwifdv qnpqevmmns sniqvkqrgp ytyrvrflak envtqdaedn tvsflqpnga |
| 121 | ifepslsvgt eadnftvlnl avaaashiyq nqfvqmilns linkskssmf qvrtlrellw |
| 181 | gyrdpflslv pypvtttvgl fypynntadg vykvfngkdn iskvaiidty kgkrnlsywe |
| 241 | shcdmingtd aasfppfvek sqvlqffssd icrsiyavfe sdvnlkgipv yrfvlpskaf |
| 301 | aspvenpdny cfctekiisk nctsygvldi skckegrpvy islphflyas pdvsepidgl |
| 361 | npneeehrty ldiepitgft lqfakrlqvn llvkpsekiq vlknlkrnyi vpilwlnetg |
| 421 | tigdekanmf rsqvtgkinl lgliemills vgvvmfvafm isycacrskt ik |
| (SEQ ID NO.: 5) | |
| MGCDRNCGLI AGAVIGAVLA VFGGILMPVG DLLIQKTIKK QVVLEEGTIA FKNWVKTGTE | |
| VYRQFWIFDV QNPQEVMMNS SNIQVKQRGP YTYRVRFLAK ENVTQDAEDN TVSFLQPNGA | |
| IFEPSLSVGT EADNFTVLNL AVAAASHIYQ NQFVQMILNS LINKSKSSMF QVRTLRELLW | |
| GYRDPFLSLV PYPVTTTVGL FYPYNNTADG VYKVFNGKDN ISKVAIIDTY KGKRNLSYWE | |
| SHCDMINGTD AASFPPFVEK SQVLQFFSSD ICRSIYAVFE SDVNLKGIPV YRFVLPSKAF ASP- | |
| VENPDNY CFCTEKIISK NCTSYGVLDI SKCKEGRPVY ISLPHFLYAS PDVSEPIDGL NPNEEE- | |
| HRTY LDIEPITGFT LQFAKRLQVN LLVKPSEKIQ VLKNLKRNYI VPILWLNETG TIGDEKANMF | |
| RSQVTGKINL LGLIEMILLS VGVVMFVAFM ISYCACRSKT IK | |
| ACCESSION NP_000063 (VERSION NP_000063.2) | |
| (SEQ ID NO.: 6) | |
| ORIGIN | |
| 1 | mgcdrncgli agavigavla vfggilmpvg dlliqktikk qvvleegtia fknwvktgte |
| 61 | vyrqfwifdv qnpqevmmns sniqvkqrgp ytyrvrflak envtqdaedn tvsflqpnga |
| 121 | ifepslsvgt eadnftvlnl avaaashiyq nqfvqmilns linkskssmf qvrtlrellw |
| 181 | gyrdpflslv pypvtttvgl fypynntadg vykvfngkdn iskvaiidty kgkrnlsywe |
| 241 | shcdmingtd aasfppfvek sqvlqffssd icrsiyavfe sdvnlkgipv yrfvlpskaf |
| 301 | aspvenpdny cfctekiisk nctsygvldi skckegrpvy islphflyas pdvsepidgl |
| 361 | npneeehrty ldiepitgft lqfakrlqvn llvkpsekiq vlknlkrnyi vpilwlnetg |
| 421 | tigdekanmf rsqvtgkinl lgliemills vgvvmfvafm isycacrskt ik |
| (SEQ ID NO.: 7) | |
| MGCDRNCGLI AGAVIGAVLA VFGGILMPVG DLLIQKTIKK QVVLEEGTIA FKNWVKTGTE | |
| VYRQFWIFDV QNPQEVMMNS SNIQVKQRGP YTYRVRFLAK ENVTQDAEDN TVSFLQPNGA | |
| IFEPSLSVGT EADNFTVLNL AVAAASHIYQ NQFVQMILNS LINKSKSSMF QVRTLRELLW | |
| GYRDPFLSLV PYPVTTTVGL FYPYNNTADG VYKVFNGKDN ISKVAIIDTY KGKRNLSYWE | |
| SHCDMINGTD AASFPPFVEK SQVLQFFSSD ICRSIYAVFE SDVNLKGIPV YRFVLPSKAF ASP- | |
| VENPDNY CFCTEKIISK NCTSYGVLDI SKCKEGRPVY ISLPHFLYAS PDVSEPIDGL NPNEEE- | |
| HRTY LDIEPITGFT LQFAKRLQVN LLVKPSEKIQ VLKNLKRNYI VPILWLNETG TIGDEKANMF | |
| RSQVTGKINL LGLIEMILLS VGVVMFVAFM ISYCACRSKT IK | |
| ACCESSION NP_001001547 (VERSION NP_001001547.1) | |
| (SEQ ID NO.: 8) | |
| ORIGIN | |
| 1 | mgcdrncgli agavigavla vfggilmpvg dlliqktikk qvvleegtia fknwvktgte |
| 61 | vyrqfwifdv qnpqevmmns sniqvkqrgp ytyrvrflak envtqdaedn tvsflqpnga |
| 121 | ifepslsvgt eadnftvlnl avaaashiyq nqfvqmilns linkskssmf qvrtlrellw |
| 181 | gyrdpflslv pypvtttvgl fypynntadg vykvfngkdn iskvaiidty kgkrnlsywe |
| 241 | shcdmingtd aasfppfvek sqvlqffssd icrsiyavfe sdvnlkgipv yrfvlpskaf |
| 301 | aspvenpdny cfctekiisk nctsygvldi skckegrpvy islphflyas pdvsepidgl |
| 361 | npneeehrty ldiepitgft lqfakrlqvn llvkpsekiq vlknlkrnyi vpilwlnetg |
| 421 | tigdekanmf rsqvtgkinl lgliemills vgvvmfvafm isycacrskt ik |

(SEQ ID NO.: 9)
MGCDRNCGLI AGAVIGAVLA VFGGILMPVG DLLIQKTIKK QVVLEEGTIA FKNWVKTGTE
VYRQFWIFDV QNPQEVMMNS SNIQVKQRGP YTYRVRFLAK ENVTQDAEDN TVSFLQPNGA
IFEPSLSVGT EADNFTVLNL AVAAASHIYQ NQFVQMILNS LINKSKSSMF QVRTLRELLW
GYRDPFLSLV PYPVTTTVGL FYPYNNTADG VYKVFNGKDN ISKVAIIDTY KGKRNLSYWE
SHCDMINGTD AASFPPFVEK SQVLQFFSSD ICRSIYAVFE SDVNLKGIPV YRFVLPSKAF
ASPVENPDNY CFCTEKIISK NCTSYGVLDI SKCKEGRPVY ISLPHFLYAS PDVSEPIDGL
NPNEEEHRTY LDIEPITGFT LQFAKRLQVN LLVKPSEKIQ VLKNLKRNYI VPILWLNETG
TIGDEKANMF RSQVTGKINL LGLIEMILLS VGVVMFVAFM ISYCACRSKT IK

### Sequence Identity

Functional equivalents and variants are used interchangeably herein. In one preferred embodiment of the invention there is also provided variants of CD36 and variants of fragments thereof. When being polypeptides, variants are determined on the basis of their degree of identity or their homology with a predetermined amino acid sequence, said predetermined amino acid sequence being one of SEQ ID NO: 4, SEQ ID NO: 5, SEQ ID NO: 6, SEQ ID NO: 7, SEQ ID NO: 8 or SEQ ID NO: 9 when the variant is a fragment, a fragment of any of the aforementioned amino acid sequences, respectively.

Accordingly, variants preferably have at least 91% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with the predetermined sequence.

The following terms are used to describe the sequence relationships between two or more polynucleotides: “predetermined sequence”, “comparison window”, “sequence identity”, “percentage of sequence identity”, and “substantial identity”.

A “predetermined sequence” is a defined sequence used as a basis for a sequence comparison; a predetermined sequence may be a subset of a larger sequence, for example, as a segment of a full-length DNA, transcriptional product thereof, gene sequence given in a sequence listing, such as a polynucleotide sequence of SEQ ID NO:1, SEQ ID NO: 2 or SEQ ID NO: 3 or may comprise a complete DNA or gene sequence. Generally, a predetermined sequence is at least 20 nucleotides in length, frequently at least 25 nucleotides in length, and often at least 50 nucleotides in length.

Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a predetermined sequence of at least 20 contiguous nucleotides and wherein the portion of the polynucleotide sequence in the comparison window may comprise additions or deletions (i.e., gaps) of 20 percent or less as compared to the predetermined sequence (which does not comprise additions or deletions) for optimal alignment of the two sequences.

Optimal alignment of sequences for aligning a comparison window may be conducted by the local homology algorithm of Smith and Waterman (1981) Adv. Appl. Math. 2: 482, by the homology alignment algorithm of Needleman and Wunsch (1970) J. Mol. Biol. 48: 443, by the search for similarity method of Pearson and Lipman (1988) Proc. Natl. Acad. Sci. (U.S.A.) 85: 2444, by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected.

The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a predetermined sequence over a comparison window of at least 20 nucleotide positions, frequently over a window of at least 25-50 nucleotides, wherein the percentage of sequence identity is calculated by comparing the predetermined sequence to the polynucleotide sequence which may include deletions or additions which total 20 percent or less of the predetermined sequence over the window of comparison. The predetermined sequence may be a subset of a larger sequence, for example, as a segment of the full-length SEQ ID NO:1, SEQ ID NO:2 and/or SEQ ID NO:3 polynucleotide sequence illustrated herein.

By the term “transcriptional or translational products” is meant herein products of gene transcription, such as a RNA transcript, for example an unspliced RNA transcript, a mRNA transcript and said mRNA transcript splicing products, and products of gene translation, such as polypeptide(s) translated from any of the gene mRNA transcripts and various products of post-translational processing of said polypeptides, such as the products of post-translational proteolytic processing of the polypeptide(s) or products of various post-translational modifications of said polypeptide(s).

As used herein, the term “transcriptional product of the gene” refers to a pre-messenger RNA molecule, pre-mRNA, that contains the same sequence information (albeit that U nucleotides replace T nucleotides) as the gene, or mature messenger RNA molecule, mRNA, which was produced due to splicing of the pre-mRNA, and is a template for translation of genetic information of the gene into a protein.

As used herein, the term “translational product of the gene” refers to a protein, which is encoded by the CD36 gene.

As used herein, the term “transcriptional product of the gene” refers to a transcript which is encoded by the CD36 encoding gene (SEQ ID NO:1, SEQ ID NO:2, SEQ ID NO:3). It is appreciated by the person skilled in the art that a number of isoforms of CD36 is known. Isoforms are versions of a protein with some small differences, usually a splice variant or the product of some posttranslational modification. The present invention relates to any isoform of CD36. The examples given herein are not meant to be limiting to the scope of the present invention.

Thus the present invention relates to methods for classifying, diagnosing and/or monitoring the atherosclerotic plaques in an individual by for example determining the concentration of at least one CD36 transcript corresponding to the transcriptional product of SEQ ID NO:1, SEQ ID NO:2, and/or SEQ ID NO:3, or part thereof.

In particular, the invention relates to methods involving the steps of determining the concentration of a CD36 encoding nucleic acid molecule, being the transcriptional products of the CD36 encoding gene in
(i) a nucleic acid sequence identified in the invention as SEQ ID NO:1, SEQ ID NO:2, and/or SEQ ID NO:3 or fragments thereof,
(ii) a nucleic acid sequence having at least 90% identity with SEQ ID NO:1, SEQ ID NO:2, and/or SEQ ID NO:3 or fragments thereof,
(iii) a nucleic acid sequence complementary to any of the sequences of (i) or (ii),

In particular, the invention relates to methods involving the steps of determining the concentration of a CD36 encoding nucleic acid molecule, being the transcriptional products of the CD36 encoding gene in

The invention also relates to methods involving the step of determining the concentration of a CD36 polypeptide or part thereof in
(i) variant proteins corresponding to the protein identified as SEQ ID NO: 4, SEQ ID NO: 5, SEQ ID NO: 6, SEQ ID NO: 7, SEQ ID NO: 8 or SEQ ID NO: 9 or variants, or fragments thereof,
(ii) polypeptide sequences having at least 90% identity with the variant proteins, or fragments thereof, of (i),

Thus, it is an embodiment of the invention to use the above identified variant proteins and/or transcriptional products for the purpose of

i) classifying atherosclerotic plaques in an individual
ii) diagnosing atherosclerotic plaques in an individual
iii) monitoring atherosclerotic plaques in an individual
iv) diagnosing an individual at risk of having and/or acquiring a symptomatic atherosclerotic plaques
v) diagnosing burden of atherosclerotic plaques in an individual
vi) diagnosing stenosis caused by atherosclerotic plaques in an individual and/or
vii) determining the treatment regime of an individual

Sequence identity is determined in one embodiment by utilising fragments of CD36 peptides comprising at least 25 contiguous amino acids and having an amino acid sequence which is at least 80%, such as 85%, for example 90%, such as 95%, for example 99% identical to the amino acid sequence of SEQ ID NO: 4, SEQ ID NO: 5, SEQ ID NO: 6, SEQ ID NO: 7, SEQ ID NO: 8 or SEQ ID NO: 9, wherein the percent identity is determined with the algorithm GAP, BESTFIT, or FASTA in the Wisconsin Genetics Software Package Release 7.0, using default gap weights.

### Conservative Amino Acid Substitutions:

Substitutions within the groups of amino acids, shown below, are considered conservative amino acid substitutions. Substitutions between the different groups of amino acids are considered non-conservative amino acid substitutions.

P, A, G, S, T (neutral, weakly hydrophobic)
Q, N, E, D, B, Z (hydrophilic, acid amine)
H, K, R (hydrophilic, basic)
F, Y, W (hydrophobic, aromatic)
L, I, V, M (hydrophobic)
C (cross-link forming)

### Individuals

The present invention relates to individuals, for example mammals and in particular humans of any sex or age. The individuals are asymptomatic with regard to the diseases caused by artherosclerosis as described herein. Thus, the individual of the present invention may never have suffered from any of atherosclerosis-related diseases. However, it is also within the scope of the present invention that the individual of the present invention may previously have been suffering from atherosclerosis-related diseases but are asymptomatic at the time for carrying out the methods of the present invention.

### Sample

The sample according to the invention may be a body fluid sample, in particular a cell-free plasma or serum sample, or a tissue sample of the plaques as such. That the plasma is cell-free may be verified microscopically following centrifugation of the blood sample. Centrifugation causes the blood cells to be separated from the plasma. Centrifugation of a blood sample in the range of 2,500 to 3000 G will result in a cell-free plasma. In one embodiment the sample is a peripheral venous blood sample. A preferred embodiment is a cell-free sample, preferably a cell-free plasma sample.

Suitable plasma preparations may be plasma from heparin-stabilised blood, Citrate-stabilised or EDTA-stabilised blood.

The samples may be fresh or frozen. For example the samples are frozen at −80° C. and are thawed only once prior to determining the concentration of CD36 and/or CD36 encoding nucleic acid.

In a particular embodiment the sample according to the invention may be a biopsy of an atherosclerotic plaque. In particular the biopsy is from a carotid atherosclerotic plaque.

### Detection Method

### Nucleic Acid Molecule Detection Methods

In the context of the present invention, “nucleic acid molecule detection methods” are understood as meaning analytical methods based on detection of DNA and/or RNA, in particular RNA in the form of mRNA, and in particular detection on high-density oligonucleotide microarrays as described below. The sequences detected are in particular the CD36 sequences disclosed above.

The nucleic acid molecule detection method is particularly useful when determining CD36 increase in a tissue sample from an atherosclerotic plaque. However, the nucleic acid molecule detection method can also be used to measure the increase in concentration of a CD36 encoding nucleic acid molecule.

It is within the general scope of the present invention to provide methods for the detection of mRNA. Such methods often involve sample extraction, PCR amplification, nucleic acid fragmentation and labeling, extension reactions, and transcription reactions.

The nucleic acid (either genomic DNA, RNA or mRNA) may be isolated from the sample according to any of a number of methods well known to those of skill in the art. One of skill will appreciate that where alterations in the copy number of a gene are to be detected genomic DNA is preferably isolated. Conversely, where an expression level of a gene such as the CD36 encoding gene is to be detected, preferably RNA (mRNA) is isolated.

For those embodiments where whole cells, or other tissue samples are being analyzed, it will typically be necessary to extract the nucleic acids from the cells or viruses, prior to continuing with the various sample preparation operations. Accordingly, following sample collection, nucleic acids may be liberated from the collected cells, viral coat etc. into a crude extract followed by additional treatments to prepare the sample for subsequent operations, such as denaturation of contaminating (DNA binding) proteins, purification, filtration and desalting.

Liberation of nucleic acids from the sample cells, and denaturation of DNA binding proteins may generally be performed by physical or chemical methods. For example, chemical methods generally employ lysing agents to disrupt the cells and extract the nucleic acids from the cells, followed by treatment of the extract with chaotropic salts such as guanidinium isothiocyanate or urea to denature any contaminating and potentially interfering proteins.

Alternatively, physical methods may be used to extract the nucleic acids and denature DNA binding proteins, such as physical protrusions within microchannels or sharp edged particles piercing cell membranes and extract their contents. Combinations of such structures with piezoelectric elements for agitation can provide suitable shear forces for lysis.

More traditional methods of cell extraction may also be used, e.g., employing a channel with restricted cross-sectional dimension which causes cell lysis when the sample is passed through the channel with sufficient flow pressure. Alternatively, cell extraction and denaturing of contaminating proteins may be carried out by applying an alternating electrical current to the sample. More specifically, the sample of cells is flowed through a microtubular array while an alternating electric current is applied across the fluid flow. Subjecting cells to ultrasonic agitation, or forcing cells through microgeometry apertures, thereby subjecting the cells to high shear stress resulting in rupture are also possible extraction methods.

Methods of isolating total mRNA are well known to those of skill in the art. RNA may be isolated from a biological sample by a variety of techniques. In one embodiment, the total nucleic acid is isolated from a given sample using, for example, an acid guanidinium-phenol-chloroform extraction method and polyA.sup. and mRNA is isolated isolated by hybridization methods using a poly(dT) matrix, which binds the polyadenylated 3′-end of mRNA species for example by oligo dT column chromatography or by using (dT)n magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)). The bound mRNAs can subsequently be washed and eluted from the matrix, and optionally precipitated. Total cellular RNA may be isolated by proteinase K treatment and DNAse treatment in an RNAse free environment followed by extraction with phenol and optionally ethanol precipitation in the presence of a salt such as sodium chloride, sodium acetate, lithium chloride or ammonium acetate.

The sample may be from tissue and/or body fluids, as defined elsewhere herein. Before analyzing the sample, e.g., on an oligonucleotide array, it will often be desirable to perform one or more sample preparation operations upon the sample. Typically, these sample preparation operations will include such manipulations as extraction of intracellular material, e.g., nucleic acids from whole cell samples, amplification of nucleic acids, fragmentation, transcription, labeling and/or extension reactions. One or more of these various operations may be readily incorporated into the present invention.

Following extraction, it will often be desirable to separate the nucleic acids from other elements of the crude extract, e.g. denatured proteins, cell membrane particles and salts. Removal of particulate matter is generally accomplished by filtration or flocculation. Further, where chemical denaturing methods are used, it may be desirable to desalt the sample prior to proceeding to the next step. Desalting of the sample and isolation of the nucleic acid may generally be carried out in a single step, e.g. by binding the nucleic acids to a solid phase and washing away the contaminating salts, or performing gel filtration chromatography on the sample passing salts through dialysis membranes. Suitable solid supports for nucleic acid binding include e.g. diatomaceous earth or silica (i.e., glass wool). Suitable gel exclusion media also well known in the art may be readily incorporated into the devices of the present invention and is commercially available from, e.g., Pharmacia and Sigma Chemical.

Alternatively, desalting methods may generally take advantage of the high electrophoretic mobility and negativity of DNA compared to other elements. Electrophoretic methods may also be utilized in the purification of nucleic acids from other cell contaminants and debris. Upon application of an appropriate electric field, the nucleic acids present in the sample will migrate toward the positive electrode and become trapped on the capture membrane. Sample impurities remaining free of the membrane are then washed away by applying an appropriate fluid flow. Upon reversal of the voltage, the nucleic acids are released from the membrane in a substantially purer form. Further, coarse filters may also be overlaid on the barriers to avoid any fouling of the barriers by particulate matter, proteins or nucleic acids, thereby permitting repeated use.

In a similar aspect, the high electrophoretic mobility of nucleic acids with their negative charges, may be utilized to separate nucleic acids from contaminants by utilizing a short column of a gel or other appropriate matrices or gels which will slow or retard the flow of other contaminants while allowing the faster nucleic acids to pass.

This invention provides nucleic acid affinity matrices that bear a large number of different nucleic acid affinity ligands allowing the simultaneous selection and removal of a large number of preselected nucleic acids from the sample. Methods of producing such affinity matrices are also provided. In general the methods involve the steps of a) providing a nucleic acid amplification template array comprising a surface to which are attached at least 50 oligonucleotides having different nucleic acid sequences, and wherein each different oligonucleotide is localized in a predetermined region of said surface, the density of said oligonucleotides is greater than about 60 different oligonucleotides per 1 cm.sup.2, and all of said different oligonucleotides have an identical terminal 3′ nucleic acid sequence and an identical terminal 5′ nucleic acid sequence. b) amplifying said multiplicity of oligonucleotides to provide a pool of amplified nucleic acids; and c) attaching the pool of nucleic acids to a solid support.

For example, nucleic acid affinity chromatography is based on the tendency of complementary, single-stranded nucleic acids to form a double-stranded or duplex structure through complementary base pairing. A nucleic acid (either DNA or RNA) can easily be attached to a solid substrate (matrix) where it acts as an immobilized ligand that interacts with and forms duplexes with complementary nucleic acids present in a solution contacted to the immobilized ligand. Unbound components can be washed away from the bound complex to either provide a solution lacking the target molecules bound to the affinity column, or to provide the isolated target molecules themselves. The nucleic acids captured in a hybrid duplex can be separated and released from the affinity matrix by denaturation either through heat, adjustment of salt concentration, or the use of a destabilizing agent such as formamide, TWEEN™-20 denaturing agent, or sodium dodecyl sulfate (SDS).

Affinity columns (matrices) are typically used either to isolate a single nucleic acid typically by providing a single species of affinity ligand. Alternatively, affinity columns bearing a single affinity ligand (e.g. oligo dt columns) have been used to isolate a multiplicity of nucleic acids where the nucleic acids all share a common sequence (e.g. a polyA).

The type of affinity matrix used depends on the purpose of the analysis. For example, where it is desired to analyze mRNA expression levels of particular genes in a complex nucleic acid sample (e.g., total mRNA) it is often desirable to eliminate nucleic acids produced by genes that are constitutively overexpressed and thereby tend to mask gene products expressed at characteristically lower levels. Thus, in one embodiment, the affinity matrix can be used to remove a number of preselected gene products (e.g., actin, GAPDH, etc.). This is accomplished by providing an affinity matrix bearing nucleic acid affinity ligands complementary to the gene products (e.g., mRNAs or nucleic acids derived therefrom) or to subsequences thereof. Hybridization of the nucleic acid sample to the affinity matrix will result in duplex formation between the affinity ligands and their target nucleic acids. Upon elution of the sample from the affinity matrix, the matrix will retain the duplexes nucleic acids leaving a sample depleted of the overexpressed target nucleic acids.

The affinity matrix can also be used to identify unknown mRNAs or cDNAs in a sample. Where the affinity matrix contains nucleic acids complementary to every known gene (e.g., in a cDNA library, DNA reverse transcribed from an mRNA, mRNA used directly or amplified, or polymerized from a DNA template) in a sample, capture of the known nucleic acids by the affinity matrix leaves a sample enriched for those nucleic acid sequences that are unknown. In effect, the affinity matrix is used to perform a subtractive hybridization to isolate unknown nucleic acid sequences. The remaining “unknown” sequences can then be purified and sequenced according to standard methods.

The affinity matrix can also be used to capture (isolate) and thereby purify unknown nucleic acid sequences. For example, an affinity matrix can be prepared that contains nucleic acid (affinity ligands) that are complementary to sequences not previously identified, or not previously known to be expressed in a particular nucleic acid sample. The sample is then hybridized to the affinity matrix and those sequences that are retained on the affinity matrix are “unknown” nucleic acids. The retained nucleic acids can be eluted from the matrix (e.g. at increased temperature, increased destabilizing agent concentration, or decreased salt) and the nucleic acids can then be sequenced according to standard methods.

Similarly, the affinity matrix can be used to efficiently capture (isolate) a number of known nucleic acid sequences. Again, the matrix is prepared bearing nucleic acids complementary to those nucleic acids it is desired to isolate. The sample is contacted to the matrix under conditions where the complementary nucleic acid sequences hybridize to the affinity ligands in the matrix. The non-hybridized material is washed off the matrix leaving the desired sequences bound. The hybrid duplexes are then denatured providing a pool of the isolated nucleic acids. The different nucleic acids in the pool can be subsequently separated according to standard methods (e.g. gel electrophoresis).

As indicated above the affinity matrices can be used to selectively remove nucleic acids from virtually any sample containing nucleic acids (e.g. in a cDNA library, DNA reverse transcribed from an mRNA, mRNA used directly or amplified, or polymerized from a DNA template, and so forth). The nucleic acids adhering to the column can be removed by washing with a low salt concentration buffer, a buffer containing a destabilizing agent such as formamide, or by elevating the column temperature.

In a preferred embodiment, the affinity matrix is packed into a columnar casing. The sample is then applied to the affinity matrix (e.g. injected onto a column or applied to a column by a pump such as a sampling pump driven by an autosampler). The affinity matrix (e.g. affinity column) bearing the sample is subjected to conditions under which the nucleic acid probes comprising the affinity matrix hybridize specifically with complementary target nucleic acids. Such conditions are accomplished by maintaining appropriate pH, salt and temperature conditions to facilitate hybridization as discussed above.

For a number of applications, it may be desirable to extract and separate messenger RNA from cells, cellular debris, and other contaminants. As such, the device of the present invention may, in some cases, include a mRNA purification chamber or channel. In general, such purification takes advantage of the poly-A tails on mRNA. In particular and as noted above, poly-T oligonucleotides may be immobilized within a chamber or channel of the device to serve as affinity ligands for mRNA. Poly-T oligonucleotides may be immobilized upon a solid support incorporated within the chamber or channel, or alternatively, may be immobilized upon the surface(s) of the chamber or channel itself. Immobilization of oligonucleotides on the surface of the chambers or channels may be carried out by methods described herein including, e.g., oxidation and silanation of the surface followed by standard DMT synthesis of the oligonucleotides.

In operation, the lysed sample is introduced to a high salt solution to increase the ionic strength for hybridization, whereupon the mRNA will hybridize to the immobilized poly-T. The mRNA bound to the immobilized poly-T oligonucleotides is then washed free in a low ionic strength buffer. The poly-T oligonucleotides may be immobilized upon porous surfaces, e.g., porous silicon, zeolites silica xerogels, scintered particles, or other solid supports.

Following sample preparation, the sample can be subjected to one or more different analysis operations. A variety of analysis operations may generally be performed, including size based analysis using, e.g., microcapillary electrophoresis, and/or sequence based analysis using, e.g., hybridization to an oligonucleotide array.

In the latter case, the nucleic acid sample may be probed using an array of oligonucleotide probes. Oligonucleotide arrays generally include a substrate having a large number of positionally distinct oligonucleotide probes attached to the substrate. These arrays may be produced using mechanical or light directed synthesis methods which incorporate a combination of photolithographic methods and solid phase oligonucleotide synthesis methods.

The basic strategy for light directed synthesis of oligonucleotide arrays is as follows. The surface of a solid support, modified with photosensitive protecting groups is illuminated through a photolithographic mask, yielding reactive hydroxyl groups in the illuminated regions. A selected nucleotide, typically in the form of a 3′-O-phosphoramidite-activated deoxynucleoside (protected at the 5′ hydroxyl with a photosensitive protecting group), is then presented to the surface and coupling occurs at the sites that were exposed to light. Following capping and oxidation, the substrate is rinsed and the surface is illuminated through a second mask to expose additional hydroxyl groups for coupling. A second selected nucleotide (e.g., 5′-protected, 3′-O-phosphoramidite-activated deoxynucleoside) is presented to the surface. The selective deprotection and coupling cycles are repeated until the desired set of products is obtained. Since photolithography is used the process can be readily miniaturized to generate high density arrays of oligonucleotide probes. Furthermore, the sequence of the oligonucleotides at each site is known. See Pease et al. Mechanical synthesis methods are similar to the light directed methods except involving mechanical direction of fluids for deprotection and addition in the synthesis steps.

For some embodiments, oligonucleotide arrays may be prepared having all possible probes of a given length. The hybridization pattern of the target sequence on the array may be used to reconstruct the target DNA sequence. Hybridization analysis of large numbers of probes can be used to sequence long stretches of DNA or provide an oligonucleotide array which is specific and complementary to a particular nucleic acid sequence. For example, in particularly preferred aspects, the oligonucleotide array will contain oligonucleotide probes which are complementary to specific target sequences, and individual or multiple mutations of these. Such arrays are particularly useful in the diagnosis of specific disorders which are characterized by the presence of a particular nucleic acid sequence.

Following sample collection and nucleic acid extraction, the nucleic acid portion of the sample is typically subjected to one or more preparative reactions. These preparative reactions include in vitro transcription, labeling, fragmentation, amplification and other reactions. Nucleic acid amplification increases the number of copies of the target nucleic acid sequence of interest. A variety of amplification methods are suitable for use in the methods and device of the present invention, including for example, the polymerase chain reaction method or (PCR), the ligase chain reaction (LCR), self sustained sequence replication (3SR), and nucleic acid based sequence amplification (NASBA).

The latter two amplification methods involve isothermal reactions based on isothermal transcription, which produce both single stranded RNA (ssRNA) and double stranded DNA (dsDNA) as the amplification products in a ratio of approximately 30 or 100 to 1, respectively. As a result, where these latter methods are employed, sequence analysis may be carried out using either type of substrate, i.e. complementary to either DNA or RNA.

Frequently, it is desirable to amplify the nucleic acid sample prior to hybridization. One of skill in the art will appreciate that whatever amplification method is used, if a quantitative result is desired, care must be taken to use a method that maintains or controls for the relative frequencies of the amplified nucleic acids.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. The high density array may then include probes specific to the internal standard for quantification of the amplified nucleic acid.

Thus, in one embodiment, this invention provides for a method of optimizing a probe set for detection of a particular gene. Generally, this method involves providing a high density array containing a multiplicity of probes of one or more particular length(s) that are complementary to sub sequences of the mRNA transcribed by the target gene. In one embodiment the high density array may contain every probe of a particular length that is complementary to a particular mRNA. The probes of the high density array are then hybridized with their target nucleic acid alone and then hybridized with a high complexity, high concentration nucleic acid sample that does not contain the targets complementary to the probes. Thus, for example, where the target nucleic acid is an RNA, the probes are first hybridized with their target nucleic acid alone and then hybridized with RNA made from a cDNA library (e.g., reverse transcribed polyA.sup.+ mRNA) where the sense of the hybridized RNA is opposite that of the target nucleic acid (to insure that the high complexity sample does not contain targets for the probes). Those probes that show a strong hybridization signal with their target and little or no cross-hybridization with the high complexity sample are preferred probes for use in the high density arrays of this invention.

PCR amplification generally involves the use of one strand of the target nucleic acid sequence as a template for producing a large number of complements to that sequence. Generally, two primer sequences complementary to different ends of a segment of the complementary strands of the target sequence hybridize with their respective strands of the target sequence, and in the presence of polymerase enzymes and nucleoside triphosphates, the primers are extended along the target sequence. The extensions are melted from the target sequence and the process is repeated, this time with the additional copies of the target sequence synthesized in the preceding steps. PCR amplification typically involves repeated cycles of denaturation, hybridization and extension reactions to produce sufficient amounts of the target nucleic acid. The first step of each cycle of the PCR involves the separation of the nucleic acid duplex formed by the primer extension. Once the strands are separated, the next step in PCR involves hybridizing the separated strands with primers that flank the target sequence. The primers are then extended to form complementary copies of the target strands. For successful PCR amplification, the primers are designed so that the position at which each primer hybridizes along a duplex sequence is such that an extension product synthesized from one primer, when separated from the template (complement), serves as a template for the extension of the other primer. The cycle of denaturation, hybridization, and extension is repeated as many times as necessary to obtain the desired amount of amplified nucleic acid.

In PCR methods, strand separation is normally achieved by heating the reaction to a sufficiently high temperature for a sufficient time to cause the denaturation of the duplex but not to cause an irreversible denaturation of the polymerase. Typical heat denaturation involves temperatures ranging from about 80.degree. C. to 105.degree. C. for times ranging from seconds to minutes. Strand separation, however, can be accomplished by any suitable denaturing method including physical, chemical, or enzymatic means. Strand separation may be induced by a helicase, for example, or an enzyme capable of exhibiting helicase activity.

In addition to PCR and IVT reactions, the methods and devices of the present invention are also applicable to a number of other reaction types, e.g., reverse transcription, nick translation, and the like.

The nucleic acids in a sample will generally be labeled to facilitate detection in subsequent steps. Labeling may be carried out during the amplification, in vitro transcription or nick translation processes. In particular, amplification, in vitro transcription or nick translation may incorporate a label into the amplified or transcribed sequence, either through the use of labeled primers or the incorporation of labeled dNTPs into the amplified sequence.

Hybridization between the sample nucleic acid and the oligonucleotide probes upon the array is then detected, using, e.g., epifluorescence confocal microscopy. Typically, sample is mixed during hybridization to enhance hybridization of nucleic acids in the sample to nucleoc acid probes on the array.

In some cases, hybridized oligonucleotides may be labeled following hybridization. For example, where biotin labeled dNTPs are used in, e.g. amplification or transcription, streptavidin linked reporter groups may be used to label hybridized complexes. Such operations are readily integratable into the systems of the present invention. Alternatively, the nucleic acids in the sample may be labeled following amplification. Post amplification labeling typically involves the covalent attachment of a particular detectable group upon the amplified sequences. Suitable labels or detectable groups include a variety of fluorescent or radioactive labeling groups well known in the art. These labels may also be coupled to the sequences using methods that are well known in the art.

Methods for detection depend upon the label selected. A fluorescent label is preferred because of its extreme sensitivity and simplicity. Standard labeling procedures are used to determine the positions where interactions between a sequence and a reagent take place. For example, if a target sequence is labeled and exposed to a matrix of different probes, only those locations where probes do interact with the target will exhibit any signal. Alternatively, other methods may be used to scan the matrix to determine where interaction takes place. Of course, the spectrum of interactions may be determined in a temporal manner by repeated scans of interactions which occur at each of a multiplicity of conditions. However, instead of testing each individual interaction separately, a multiplicity of sequence interactions may be simultaneously determined on a matrix.

Means of detecting labeled target (sample) nucleic acids hybridized to the probes of the high density array are known to those of skill in the art. Thus, for example, where a colorimetric label is used, simple visualization of the label is sufficient. Where a radioactive labeled probe is used, detection of the radiation (e.g with photographic film or a solid state detector) is sufficient.

In a preferred embodiment, however, the target nucleic acids are labeled with a fluorescent label and the localization of the label on the probe array is accomplished with fluorescent microscopy. The hybridized array is excited with a light source at the excitation wavelength of the particular fluorescent label and the resulting fluorescence at the emission wavelength is detected. In a particularly preferred embodiment, the excitation light source is a laser appropriate for the excitation of the fluorescent label.

The target polynucleotide may be labeled by any of a number of convenient detectable markers. A fluorescent label is preferred because it provides a very strong signal with low background. It is also optically detectable at high resolution and sensitivity through a quick scanning procedure. Other potential labeling moieties include, radioisotopes, chemiluminescent compounds, labeled binding proteins, heavy metal atoms, spectroscopic markers, magnetic labels, and linked enzymes.

Another method for labeling may bypass any label of the target sequence. The target may be exposed to the probes, and a double strand hybrid is formed at those positions only. Addition of a double strand specific reagent will detect where hybridization takes place. An intercalative dye such as ethidium bromide may be used as long as the probes themselves do not fold back on themselves to a significant extent forming hairpin loops. However, the length of the hairpin loops in short oligonucleotide probes would typically be insufficient to form a stable duplex.

Suitable chromogens will include molecules and compounds which absorb light in a distinctive range of wavelengths so that a color may be observed, or emit light when irradiated with radiation of a particular wave length or wave length range, e.g., fluorescers. Biliproteins, e.g., phycoerythrin, may also serve as labels.

A wide variety of suitable dyes are available, being primarily chosen to provide an intense color with minimal absorption by their surroundings. Illustrative dye types include quinoline dyes, triarylmethane dyes, acridine dyes, alizarine dyes, phthaleins, insect dyes, azo dyes, anthraquinoid dyes, cyanine dyes, phenazathionium dyes, and phenazoxonium dyes.

A wide variety of fluorescers may be employed either by themselves or in conjunction with quencher molecules. Fluorescers of interest fall into a variety of categories having certain primary functionalities. These primary functionalities include 1- and 2-aminonaphthalene, p,p′-diaminostilbenes, pyrenes, quaternary phenanthridine salts, 9-aminoacridines, p,p′-diaminobenzophenone imines, anthracenes, oxacarbocyanine, merocyanine, 3-aminoequilenin, perylene, bis-benzoxazole, bis-p-oxazolyl benzene, 1,2-benzophenazin, retinol, bis-3-aminopyridinium salts, hellebrigenin, tetracycline, sterophenol, benzimidzaolylphenylamine, 2-oxo-3-chromen, indole, xanthen, 7-hydroxycoumarin, phenoxazine, salicylate, strophanthidin, porphyrins, triarylmethanes and flavin. Individual fluorescent compounds which have functionalities for linking or which can be modified to incorporate such functionalities include, e.g., dansyl chloride; fluoresceins such as 3,6-dihydroxy-9-phenylxanthhydrol; rhodamineisothiocyanate; N-phenyl 1-amino-8-sulfonatonaphthalene; N-phenyl 2-amino-6-sulfonatonaphthalene; 4-acetamido-4-isothiocyanato-stilbene-2,2′-disulfonic acid; pyrene-3-sulfonic acid; 2-toluidinonaphthalene-6-sulfonate; N-phenyl, N-methyl 2-aminoaphthalene-6-sulfonate; ethidium bromide; stebrine; auromine-0,2-(9′-anthroyl)palmitate; dansyl phosphatidylethanolamine; N,N′-dioctadecyl oxacarbocyanine; N,N′-dihexyl oxacarbocyanine; merocyanine, 4-(3′ pyrenyl)butyrate; d-3-aminodesoxy-equilenin; 12-(9′-anthroyl)stearate; 2-methylanthracene; 9-vinylanthracene; 2,2′-(vinylene-p-phenylene)bisbenzoxazole; p-bis>2-(4-methyl-5-phenyl-oxazolyl)!benzene; 6-dimethylamino-1,2-benzophenazin; retinol; bis(3′-aminopyridinium) 1,10-decandiyl diiodide; sulfonaphthylhydrazone of hellibrienin; chlorotetracycline; N-(7-dimethylamino-4-methyl-2-oxo-3-chromenyl)maleimide; N->p-(2-benzimidazolyl)-phenyl!maleimide; N-(4-fluoranthyl)maleimide; bis(homovanillic acid); resazarin; 4-chloro-7-nitro-2,1,3-benzooxadiazole; merocyanine 540; resorufin; rose bengal; and 2,4-diphenyl-3(2H)-furanone.

Desirably, fluorescers should absorb light above about 300 nm, preferably about 350 nm, and more preferably above about 400 nm, usually emitting at wavelengths greater than about 10 nm higher than the wavelength of the light absorbed. It should be noted that the absorption and emission characteristics of the bound dye may differ from the unbound dye. Therefore, when referring to the various wavelength ranges and characteristics of the dyes, it is intended to indicate the dyes as employed and not the dye which is unconjugated and characterized in an arbitrary solvent. Fluorescers are generally preferred because by irradiating a fluorescer with light, one can obtain a plurality of emissions. Thus, a single label can provide for a plurality of measurable events.

Detectable signal may also be provided by chemiluminescent and bioluminescent sources. Chemiluminescent sources include a compound which becomes electronically excited by a chemical reaction and may then emit light which serves as the detectible signal or donates energy to a fluorescent acceptor. A diverse number of families of compounds have been found to provide chemiluminescence under a variety of conditions. One family of compounds is 2,3-dihydro-1,-4-phthalazinedione. The most popular compound is luminol, which is the 5-amino compound. Other members of the family include the 5-amino-6,7,8-trimethoxy- and the dimethylamino>ca!benz analog. These compounds can be made to luminesce with alkaline hydrogen peroxide or calcium hypochlorite and base. Another family of compounds is the 2,4,5-triphenylimidazoles, with lophine as the common name for the parent product. Chemiluminescent analogs include para-dimethylamino and -methoxy substituents. Chemiluminescence may also be obtained with oxalates, usually oxalyl active esters, e.g., p-nitrophenyl and a peroxide, e.g., hydrogen peroxide, under basic conditions. Alternatively, luciferins may be used in conjunction with luciferase or lucigenins to provide bioluminescence.

Spin labels are provided by reporter molecules with an unpaired electron spin which can be detected by electron spin resonance (ESR) spectroscopy. Exemplary spin labels include organic free radicals, transitional metal complexes, particularly vanadium, copper, iron, and manganese, and the like. Exemplary spin labels include nitroxide free radicals.

In addition, amplified sequences may be subjected to other post amplification treatments. For example, in some cases, it may be desirable to fragment the sequence prior to hybridization with an oligonucleotide array, in order to provide segments which are more readily accessible to the probes, which avoid looping and/or hybridization to multiple probes. Fragmentation of the nucleic acids may generally be carried out by physical, chemical or enzymatic methods that are known in the art.

Following the various sample preparation operations, the sample will generally be subjected to one or more analysis operations. Particularly preferred analysis operations include, e.g. sequence based analyses using an oligonucleotide array and/or size based analyses using, e.g. microcapillary array electrophoresis.

In some embodiments it may be desirable to provide an additional, or alternative means for analyzing the nucleic acids from the sample

Microcapillary array electrophoresis generally involves the use of a thin capillary or channel which may or may not be filled with a particular separation medium. Electrophoresis of a sample through the capillary provides a size based separation profile for the sample. Microcapillary array electrophoresis generally provides a rapid method for size based sequencing, PCR product analysis and restriction fragment sizing. The high surface to volume ratio of these capillaries allows for the application of higher electric fields across the capillary without substantial thermal variation across the capillary, consequently allowing for more rapid separations. Furthermore, when combined with confocal imaging methods these methods provide sensitivity in the range of attomoles, which is comparable to the sensitivity of radioactive sequencing methods.

In many capillary electrophoresis methods, the capillaries e.g. fused silica capillaries or channels etched, machined or molded into planar substrates, are filled with an appropriate separation/sieving matrix. Typically, a variety of sieving matrices are known in the art may be used in the microcapillary arrays. Examples of such matrices include, e.g. hydroxyethyl cellulose, polyacrylamide and agarose. Gel matrices may be introduced and polymerized within the capillary channel. However, in some cases this may result in entrapment of bubbles within the channels which can interfere with sample separations. Accordingly, it is often desirable to place a preformed separation matrix within the capillary channel(s), prior to mating the planar elements of the capillary portion. Fixing the two parts, e.g. through sonic welding, permanently fixes the matrix within the channel. Polymerization outside of the channels helps to ensure that no bubbles are formed. Further, the pressure of the welding process helps to ensure a void-free system.

In addition to its use in nucleic acid “fingerprinting” and other sized based analyses the capillary arrays may also be used in sequencing applications. In particular, gel based sequencing techniques may be readily adapted for capillary array electrophoresis.

Also reverse transcription PCR can be employed in the present invent to detect the concentration of transcripts of the CD36 encoding gene. RT-PCR, called the “first strand reaction,” complementary DNA is made from a messenger RNA template using dNTPs and an RNA-dependent DNA polymerase (reverse transcriptase) through the process of reverse transcription. RT-PCR exploits a characteristic of mature mRNAs known as the 3′ polyadenylated region, commonly called the poly(A) tail, as a common binding site for poly(T) DNA primers. In the case of bacterial mRNA, which lack a poly(A) tail sequence-specific primers can be generated to amplify the target mRNA sequence. These primers will anneal to the 3′ end of every mRNA in the solution, allowing 5′->3′ synthesis of complementary DNA by the reverse transcriptase enzyme. cDNA can also be prepared from mRNA by using gene specific primer or random hexamer primers.

After the reverse transcriptase reaction is complete, and complementary DNA has been generated from the original single-stranded mRNA, standard polymerase chain reaction, termed the “second strand reaction,” is initiated. If the initial mRNA templates were derived from the same tissue, subsequent PCR reactions can be used to probe the cDNA library that was created by reverse transcription. Primers can be designed to amplify target genes being expressed in the source tissue. Quantitative real-time PCR can then be used to compare levels of gene expression.

In the scope of the present invention the term “hybridization” signifies hybridization under conventional hybridization conditions, preferably under stringent conditions, as described for example in Sambrook et al., Molecular Cloning, A Laboratory Manual, 2nd Edition (1989) Cold Spring Harbor Laboratory Press, Cold Spring Harbor, N.Y.). The term “stringent” when used in conjunction with hybridization conditions is as defined in the art, i.e. 15-20° C. under the melting point Tₘ, cf. Sambrook et al, 1989, pages 11.45-11.49. Preferably, the conditions are “highly stringent”, i.e. 5-10° C. under the melting point Tₘ. Under highly stringent conditions hybridization only occurs if the identity between the oligonucleotide sequence and the locus of interest is 100%, while no hybridization occurs if there is just one mismatch between oligonucleotide and DNA locus. Such optimised hybridization results are reached by adjusting the temperature and/or the ionic strength of the hybridization buffer as described in the art. However, equally high specificity may be obtained using high-affinity DNA analogues. One such high-affinity DNA analogue has been termed “locked nucleic acid” (LNA). LNA is a novel class of bicyclic nucleic acid analogues in which the furanose ring conformation is restricted in by a methylene linker that connects the 2′-O position to the 4′-C position. Common to all of these LNA variants is an affinity toward complementary nucleic acids, which is by far the highest affinity reported for a DNA analogue (Ørum et al. (1999) Clinical Chemistry 45, 1898-1905; WO 99/14226 EXIQON). LNA probes are commercially available from Proligo LLC, Boulder, Colo., USA. Another high-affinity DNA analogue is the so-called protein nucleic acid (PNA). In PNA compounds, the sugar backbone of an oligonucleotide is replaced with an amide containing backbone, in particular an aminoethylglycine backbone. The nucleobases are retained and are bound directly or indirectly to aza nitrogen atoms of the amide portion of the backbone (Science (1991) 254: 1497-1500).

Various different labels can be coupled to the probe. Among these fluorescent reporter groups are preferred because they result in a high signal/noise ratio.

Suitable examples of the fluorescent group include fluorescein, Cy2, Cy3, Cy3.5, Cy5, Cy5.5, Cy7, acridin, Hoechst 33258, Rhodamine, Rhodamine Green, Tetramethylrhodamine, Texas Red, Cascade Blue, Oregon Green, Alexa Fluor, europium and samarium.

Another type of labels is enzyme tags. After hybridization to the target nucleic acid sequence a substrate for the enzyme is added and the formation of a coloured product is measured. Examples of enzyme tags include a beta-Galactosidase, a peroxidase, horseradish peroxidase, a urease, a glycosidase, alkaline phosphatase, chloramphenicol acetyltransferase and a luciferase.

A further group of labels include chemiluminescent group, such as hydrazides such as luminol and oxalate esters.

A still further possibility is to use a radioisotope and detect the hybrid using scintillation counting. The radioisotope may be selected from the group consisting of ³²P, ³³P, ³⁵S, ¹²⁵I, ⁴⁵Ca, ¹⁴C and ³H.

One particularly preferred embodiment of the probe based detection comprises the use of a capture probe for capturing a target nucleic acid sequence. The capture probe is bound to a solid surface such as a bead, a well or a stick. The captured target nucleic acid sequence can then be contacted with the detection probe under conditions of high stringency and the allele can be detected.

One embodiment of the probe based technique based on TAQMAN technique. This is a method for measuring PCR product accumulation using a dual-labeled fluorogenic oligonucleotide probe called a TAQMAN® probe. This probe is composed of a short (ca. 20-25 bases) oligodeoxynucleotide that is labeled with two different fluorescent dyes. On the 5′ terminus is a reporter dye and on the 3′ terminus is a quenching dye. This oligonucleotide probe sequence is homologous to an internal target sequence present in the PCR amplicon. When the probe is intact, energy transfer occurs between the two fluorophors and emission from the reporter is quenched by the quencher. During the extension phase of PCR, the probe is cleaved by 5′ nuclease activity of Taq polymerase thereby releasing the reporter from the oligonucleotide-quencher and producing an increase in reporter emission intensity.

Other suitable methods include using mass spectrometry, single base extension, determining the Tm profile of a hybrid between a probe and a target nucleic acid sequence, using single strand conformation polymorphism, using single strand conformation polymorphism heteroduplex, using RFLP or RAPD, using HPLC, using sequencing of a target nucleic acid sequence from said biological sample.

### Oligonucleotide Primer and or Probe

In one aspect the present invention relates to oligonucleotide primers and/or probes for detecting a CD36 encoding nucleic acid molecule or a part thereof, in particular for detecting a CD36 transcriptional product or part thereof, wherein said at least one nucleotide primer and/or probe detects at least one of the CD36 transcripts or part thereof.

As known to the skilled person an isolated oligonucleotide primer of the present invention is a nucleic acid molecule sufficiently complementary to the sequence on which it is based and of sufficiently length to selectively hybridise to the corresponding region of a nucleic acid molecule intended to be amplified. The primer is able to prime the synthesis of the corresponding region of the intended nucleic acid molecule in the methods described above. Similarly, an isolated oligonucleotide probe of the present invention is a molecule for example a nucleic acid molecule of sufficient length and sufficiently complementary to the nucleic acid sequence of interest which selectively binds to the nucleic acid sequence of interest under high or low stringency conditions. It is thus understood that the at least one oligonucleotide primer and/or probe is able to amplify the CD36 encoding nucleic acid molecule or part thereof. It is further appreciated by the skilled person that at least one oligonucleotide primer and/or probe is used, such as 3, 4, 5, 6, 7, 8, 9, 10, 15, 20, 25 oligonucleotide primer and/or probes is used according to the present invention.

In one embodiment the invention relates to an isolated oligonucleotide comprising at least 10 contiguous nucleotides being 100% identical to a subsequence of the CD36 transcript or part thereof. As explained elsewhere herein such probes may be used for determining the concentration of at least one CD36 transcript or part thereof. Preferably the isolated oligonucleotide comprises at least 10 contiguous bases of a sequence identified as SEQ ID NOs: 10-45 or the corresponding complementary strand, or a strand sharing at least 90% sequence identity more preferably at least 95% sequence identity with SEQ ID NOs: 10-45 or a complementary strand thereof, said isolated oligonucleotide comprising a genetic marker of the invention.

3 probe sets A, B and C were used in the detection according to the present invention.

| PROBE SET A | | | | |
|---|---|---|---|---|
| SEQ ID NO: 10 | | | | |
| >HG-U133A_2:206488_S_AT (target sequence) | | | | |
| aactggattcactttacaatttgcaaaacggctgcaggtcaacctattggtcaagccatc | | | | |
| agaaaaaattcaagtattaaagaatctgaagaggaactatattgtgcctattctttggct | | | | |
| taatgagactgggaccattggtgatgagaaggcaaacatgttcagaagtcaagtaactgg | | | | |
| aaaaataaacctccttggcctgatagaaatgatcttactcagtgttggtgtggtgatgtt | | | | |
| tgttgcttttatgatttcatattgtgcatgcagatcg | | | | |
| Reference: https://www.affymetrix.com/analysis/netaffx/fullrecord.affx?pk=HG- | | | | |
| U133A 2%3A206488 S AT | | | | |
| Probe Seq (5′-3′): | Probe X: | Probe Y: | Position: | Target Strandedness |
| SEQ ID NO: 11: AACTGGATTCACTTTACAATTTGCA | 145 | 131 | 1272 | Antisense |
| SEQ ID NO: 12: GCAAAACGGCTGCAGGTCAACCTAT | 11 | 323 | 1294 | Antisense |
| SEQ ID NO: 13: GTCAACCTATTGGTCAAGCCATCAG | 227 | 443 | 1309 | Antisense |
| SEQ ID NO: 14: GGAACTATATTGTGCCTATTCTTTG | 662 | 515 | 1364 | Antisense |
| SEQ ID NO: 15: GTGCCTATTCTTTGGCTTAATGAGA | 553 | 455 | 1375 | Antisense |
| SEQ ID NO: 16: AATGAGACTGGGACCATTGGTGATG | 242 | 151 | 1393 | Antisense |
| SEQ ID NO: 17: TGGAAAAATAAACCTCCTTGGCCTG | 517 | 535 | 1449 | Antisense |
| SEQ ID NO: 18: CCTCCTTGGCCTGATAGAAATGATC | 686 | 259 | 1461 | Antisense |
| SEQ ID NO: 19: GATCTTACTCAGTGTTGGTGTGGTG | 46 | 409 | 1482 | Antisense |
| SEQ ID NO: 20: TTGGTGTGGTGATGTTTGTTGCTTT | 127 | 689 | 1496 | Antisense |
| SEQ ID NO: 21: GATTTCATATTGTGCATGCAGATCG | 171 | 413 | 1524 | Antisense |

| PROBE SET B | | | | |
|---|---|---|---|---|
| SEQ ID NO: 22 | | | | |
| >HG-U133A_2:209555_S_AT (target sequence) | | | | |
| aaccaatttgtgttgttctgattcaataattggtttctgggtggccaattcagaagaaga | | | | |
| gtgtacatgctcaacagtctccaggaccatcagtatactgcatttcatgtgcaccaaata | | | | |
| ttttgaaagacatttataaataattggcttatgactcatatttctctatgaataccttca | | | | |
| tacagcaggtataactcttttctttatgggcttaaatattttgtcactgatcctgcaaat | | | | |
| ggacatcattttagcacactagcggtttatattttaaggaccttcattctctgttctgca | | | | |
| cctcttctggaaattgagtaaattttgctttttttttttactcagttgcaacttacgctt | | | | |
| ggcatcttcagaatgcttttctagcattaagagatgtaaatgataaaggaattattgtat | | | | |
| gaaatattacaaagcgtagactatgcattgttattcattataatattttttgctgtcata | | | | |
| atcgcctcata | | | | |
| Reference: https://www.affymetrix.com/analysis/netaffx/fullrecord.affx?pk=HG- | | | | |
| U133A 2%3A209555 S AT | | | | |
| Probe Seq (5′-3′): | Probe X: | Probe Y: | Position: | Target Strandedness |
| SEQ ID NO: 23: AACCAATTTGTGTTGTTCTGATTCA | 586 | 135 | 1702 | Antisense |
| SEQ ID NO: 24: TTGGTTTCTGGGTGGCCAATTCAGA | 119 | 677 | 1731 | Antisense |
| SEQ ID NO: 25: AACAGTCTCCAGGACCATCAGTATA | 22 | 117 | 1774 | Antisense |
| SEQ ID NO: 26: GTATACTGCATTTCATGTGCACCAA | 663 | 431 | 1794 | Antisense |
| SEQ ID NO: 27: GTCACTGATCCTGCAAATGGACATC | 499 | 443 | 1924 | Antisense |
| SEQ ID NO: 28: ATGGACATCATTTTAGCACACTAGC | 454 | 39 | 1940 | Antisense |
| SEQ ID NO: 29: AGCACACTAGCGGTTTATATTTTAA | 624 | 71 | 1954 | Antisense |
| SEQ ID NO: 30: TTTAAGGACCTTCATTCTCTGTTCT | 675 | 655 | 1974 | Antisense |
| SEQ ID NO: 31: CAGTTGCAACTTACGCTTGGCATCT | 139 | 187 | 2044 | Antisense |
| SEQ ID NO: 32: GCTTGGCATCTTCAGAATGCTTTTC | 667 | 311 | 2058 | Antisense |
| SEQ ID NO: 33: TTTTTGCTGTCATAATCGCCTCATA | 683 | 659 | 2168 | Antisense |

| PROBE SET C: | | | | |
|---|---|---|---|---|
| SEQ ID NO: 34: | | | | |
| >HG-U133A_2:209554_AT (target sequence) | | | | |
| gaaatctgggcttggatgtgtgtgtgtgtgtgtgtgtgtgtgtgtgcacatatgcagtgt | | | | |
| ggtgggagtggggcaacttggggaatatgttacatgtgtgactgtgttggccctggcgaa | | | | |
| gtaatgtgtcagaaagggtaaatgtgtggacacttgcaagtgctcatggatgaatttata | | | | |
| tgtttgagtcatagaaaaagtgtaccctttgatagaagcacattttctttccaaaggcgt | | | | |
| ggttattaaccagcagaattatagcaggtatacataacttaagtg | | | | |
| Reference: | | | | |
| https://www.affymetrix.com/analysis/netaffx/fullrecord.affx?pk=HG- | | | | |
| U133A 2%3A209554 AT | | | | |
| Probe Seq (5′-3′): | Probe X: | Probe Y: | Position: | Target Strandedness |
| SEQ ID NO: 35: GAAATCTGGGCTTGGATGTGTGTGT | 350 | 357 | 3352 | Antisense |
| SEQ ID NO: 36: GTGTGCACATATGCAGTGTGGTGGG | 327 | 465 | 3393 | Antisense |
| SEQ ID NO: 37: GTGTGGTGGGAGTGGGGCAACTTGG | 394 | 467 | 3408 | Antisense |
| SEQ ID NO: 38: GAGTGGGGCAACTTGGGGAATATGT | 644 | 391 | 3417 | Antisense |
| SEQ ID NO: 39: GTTACATGTGTGACTGTGTTGGCCC | 289 | 425 | 3440 | Antisense |
| SEQ ID NO: 40: GTTGGCCCTGGCGAAGTAATGTGTC | 602 | 417 | 3457 | Antisense |
| SEQ ID NO: 41: GTGTGGACACTTGCAAGTGCTCATG | 314 | 467 | 3495 | Antisense |
| SEQ ID NO: 42: ACTTGCAAGTGCTCATGGATGAATT | 10 | 101 | 3503 | Antisense |
| SEQ ID NO: 43: AGTGTACCCTTTGATAGAAGCACAT | 137 | 51 | 3550 | Antisense |
| SEQ ID NO: 44: AAGGCGTGGTTATTAACCAGCAGAA | 277 | 149 | 3585 | Antisense |
| SEQ ID NO: 45: ATAGCAGGTATACATAACTTAAGTG | 181 | 1 | 3612 | Antisense |

Further preferred isolated oligonucleotides may comprise at least 10 contiguous bases of any of the sequence identified as SEQ ID NO:1, SEQ ID NO:2 or SEQ ID NO:3 or the corresponding complementary strand thereof, or a strand sharing at least 90% sequence identity more preferably at least 95% sequence identity with the SEQ ID NO:1, SEQ ID NO:2 or SEQ ID NO:3, or a complementary strand thereof, said isolated oligonucleotide comprising a polymorphism of the invention.

The length of the isolated oligonucleotide depends on the purpose. When being used for amplification from a sample of genomic DNA, the length of the primers should be at least 15 and more preferably even longer to ensure specific amplification of the desired target nucleotide sequence. When being used for amplification from mRNA the length of the primers can be shorter while still ensuring specific amplification. In one particular embodiment one of the pair of primers may be an allele specific primer in which case amplification only occurs if the specific allele is present in the sample. When the isolated oligonucleotides are used as hybridisation probes for detection, the length is preferably in the range of 10-15 nucleotides. This is enough to ensure specific hybridisation in a sample with an amplified target nucleic acid sequence. When using nucleotides which bind stronger than DNA (e.g. LNA and/or PNA), the length of the probe can be somewhat shorter, e.g. down to 7-8 bases.

The length may be at least 15 contiguous nucleotides, such as at least 20 nucleotides. An upper limit preferably determines the maximum length of the isolated oligonucleotide. Accordingly, the isolated oligonucleotide may be less than 1000 nucleotides, more preferably less than 500 nucleotides, more preferably less than 100 nucleotides, such as less than 75 nucleotides, for example less than 50 nucleotides, such as less than 40 nucleotides, for example less than 30 nucleotides, such as less than 20 nucleotides.

The isolated oligonucleotide may comprise from 10 to 50 nucleotides, such as from 10 to 15, from 15 to 20, from 20 to 25, or comprising from 20 to 30 nucleotides, or from 15 to 25 nucleotides.

Depending on the use the polymorphism may be located in the centre of the nucleic acid sequence, in the 5′ end of the nucleic acid sequence, or in the 3′ end of the nucleic acid sequence. For detection based on single base extension the sequence of the oligonucleotide is adjacent to the mutation/polymorphism, either in the 3′ or 5′ direction.

The isolated oligonucleotide sequence may be complementary to a sub-sequence of the coding strand of a target nucleotide sequence or to a sub-sequence to the non-coding strand of a target nucleotide sequence as the polymorphism may be assessed with similar efficiency in the coding and the non-coding strand.

The isolated oligonucleotide sequence may be made from RNA, DNA, LNA, PNA monomers or from chemically modified nucleotides capable of hybridising to a target nucleic acid sequence. The oligonucleotides may also be made from mixtures of said monomers.

A general term for primers and probes of is the term ‘oligonucleotide’ which comprises oligonucleotides of both natural and/or non-natural nucleotides, including any combination thereof. The natural and/or non-natural nucleotides may be linked by natural phosphodiester bonds or by non-natural bonds. Oligonucleotide is used interchancably with polynucleotide. The oligomer or polymer sequences of the present invention are formed from the chemical or enzymatic addition of monomer subunits. The term “oligonucleotide” as used herein includes linear oligomers of natural or modified monomers or linkages, including deoxyribonucleotides, ribonucleotides, anomeric forms thereof, peptide nucleic acid monomers (PNAs), locked nucleotide acid monomers (LNA), and the like, capable of specifically binding to a single stranded polynucleotide tag by way of a regular pattern of monomer-to-monomer interactions, such as Watson-Crick type of base pairing, base stacking, Hoogsteen or reverse Hoogsteen types of base pairing, or the like. Usually monomers are linked by phosphodiester bonds or analogs thereof to form oligonucleotides ranging in size from a few monomeric units, e.g. 3-4, to several tens of monomeric units, e.g. 40-60. Whenever an oligonucleotide is represented by a sequence of letters, such as “ATGCCTG,” it will be understood that the nucleotides are in 5′→3′ order from left to right and the “A” denotes deoxyadenosine, “C” denotes deoxycytidine, “G” denotes deoxyguanosine, and “T” denotes thymidine, unless otherwise noted. Usually oligonucleotides of the invention comprise the four natural nucleotides; however, they may also comprise methylated or non-natural nucleotide analogs. Suitable oligonucleotides may be prepared by the phosphoramidite method described by Beaucage and Carruthers (Tetrahedron Lett., 22, 1859-1862, 1981), or by the triester method according to Matteucci, et al. (J. Am. Chem. Soc., 103, 3185, 1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical configuration typically associated with, for example, DNA. In addition to the 100% complementary form of double-stranded oligonucleotides, the term “double-stranded” as used herein is also meant to refer to those forms which include such structural features as bulges and loops. For example as described in U.S. Pat. No. 5,770,722 for a unimolecular double-stranded DNA. It is clear to those skilled in the art when oligonucleotides having natural or non-natural nucleotides may be employed, e.g. where processing by enzymes is called for, usually oligonucleotides consisting of natural nucleotides are required. When nucleotides are conjugated together in a string using synthetic procedures, they are always referred to as oligonucleotides.

### Immunological Methods

In the context of the present invention, “immunological methods” are understood as meaning analytical methods based on immunochemistry, in particular on an antigen-antibody reaction. Examples of immunological methods include immunoassays such as radioimmunoassay (RIA), enzyme immunoassay (EIA, combined with solid-phase technique: ELISA (Enzyme Linked Immuno Sorbent Assay)) or else immunofluorescence assays. The immunoassay is carried out by exposing the sample to be investigated to a CD36-binding antibody and detecting and quantifying the amount of CD36 bound to this antibody. In these assays, detection and quantification is carried out directly or indirectly in a known manner. Thus, detection and quantification of the antigen-antibody complexes is made possible by using suitable labels which may be carried by the antibody directed against CD36 and/or by a secondary antibody directed against the primary antibody. Depending on the type of the abovementioned immunoassays, the labels are, for example, radioactive labels, a chemiluminescent label, fluorescent dyes or else enzymes, such as phosphatase or peroxidase, which can be detected and quantified with the aid of a suitable substrate.

In one embodiment of the invention, the immunological method is carried out with the aid of a suitable solid phase. Suitable solid phases which may be mentioned include the customary commercial microtiter plates made of polystyrene or membranes (for example made of polyvinylidene difluoride, PVDF) which are customarily used for the ELISA technique.

To carry out a process according to the invention, a suitable sample, such as a liquid patient sample is applied to the solid phase. The sample is preferably a plasma sample wherein the CD36 or fraction thereof is present in unbound form or is present in a form which could be bound to its ligand LDL. The assumption herein that sCD36 is present in complex with a high molecular fraction in plasma implies that it is preferred to freeze and thaw the samples to be tested, such that said high molecular complex (lipid-protein complex) possibly is degraded.

In one aspect the invention relates to a method, wherein the CD36 polypeptide concentration is determined by i) providing a sample to be investigated, ii) providing an anti-CD36 antibody, iii) exposing the sample to the anti-CD36 antibody, iv) optionally, exposing said CD36-antibody complex to at least a further antibody directed against said CD36-antibody complex, and v) detecting and quantifying the amount of said antibody

In a more specific aspect of the invention there is provided a method for determining human CD36 in a plasma sample by solid phase ELISA enzyme immunoassay which comprises the steps of

(i) providing a plasma sample to be investigated,
(ii) providing an anti-CD36 antibody as defined herein,
(iii) exposing the sample to be investigated to a solid phase and the antibody, and
(iv) detecting and quantifying the amount of the antibody which binds to CD36; and in a more preferred aspect of the invention there is provided a method for determining human CD36 in a plasma sample by solid phase ELISA enzyme immunoassay which comprises the steps of
(i) providing a plasma sample to be investigated,
(ii) providing an anti-CD36 antibody,
(iii) exposing the sample to be investigated to the anti-CD36 antibody bound to a solid phase,
(iv) optionally exposing the CD36-antibody complex to a second anti-CD36 antibody, and
(iv) detecting and quantifying the amount of the antibody which binds to CD36,
and wherein the solid phase preferably is a microtiter plate, the CD36 is preferably present (part of) in a high molecular weight plasma fraction said high molecular weight plasma fraction preferably being a CD36-lipoprotein complex; said anti-CD36 antibody is preferably selected from the group consisting of monoclonal antibodies as specified below; said determination is preferably carried out by a solid phase enzyme immunoassay, wherein, in the enzyme immunoassay, the sample is exposed to a first, human CD36-binding antibody, and the amount of bound CD36 is measured using a second CD36-binding antibody carrying an enzyme label, where the measurement is carried out by an enzyme-catalyzed colour reaction or chemiluminescence.

It is also within the scope of the present invention to determine the concentration of CD36 by multiplex suspension array technique.

### Antibodies

One aspect of the present invention relates to an antibody directed to an epitope of CD36 polypeptide or part thereof as described elsewhere herein. The antibody may be used in methods of the present invention relating to methods for classifying an atherosclerotic plaque, diagnosing an atherosclerotic plaque, monitoring an atherosclerotic plaque, diagnosing a risk of having and/or acquiring a symptomatic atherosclerotic plaque, diagnosing the burden of an atherosclerotic plaque, diagnosing stenosis, and/or determining the treatment regime of an individual. Thus, epitope in this context covers any epitope capable of being recognized by an antibody or a binding fragment thereof.

The term “antibody” as used herein includes both polyclonal and monoclonal antibodies, as well as fragments thereof, such as, Fv, Fab and F(ab)2 fragments that are capable of binding antigen or hapten. It includes conventional murine monoclonal antibodies as well as human antibodies, and humanized forms of non-human antibodies, and it also includes ‘antibodies’ isolated from phage antibody libraries.

The antibodies of the present invention may be polyclonal or monoclonal and may be produced by in vivo or in vitro methods known in the art. Thus, the anti-CD36 antibody is selected from the group consisting of monoclonal and polyclonal CD36-specific antibodies.

A monoclonal antibody is an antibody produced by a hybridoma cell. Methods of making monoclonal antibody-synthesizing hybridoma cells are well known to those skilled in the art, e.g, by the fusion of an antibody producing B lymphocyte with an immortalized B-lymphocyte cell line.

A polyclonal antibody is a mixture of antibody molecules (specific for a given antigen) that has been purified from an immunized (to that given antigen) animal's blood, where a non-limiting example is antibody molecules from rabbit. Such antibodies are polyclonal in that they are the products of many different populations of antibody-producing cells.

The invention also pertains to mixtures of monoclonal and/or polyclonal antibodies. Also a mixture of at least two monoclonal antibodies is within the scope of the present invention. It is appreciated that the mixture may comprise 3, 4, 5, 6, 7, 8, 9, 10, or 15 monoclonal antibodies.

The anti-CD36 antibody used herein may be a monoclonal antibody as described above, and/or an isolated polyclonal antibody, obtainable by an immunization process in which purified or recombinant human CD36 is used as antigen component and the antibody is preferably specific for various fractions of the CD36 protein which is present in human blood plasma in soluble form, optionally as a part of a CD36-lipoprotein complex, such as CD36 or a fraction thereof bound to Low Density Lipoprotein (LDL), IDL (Intermediate Density Lipoprotein or VLDL (Very Low Density Lipoprotein) which may be present in a high molecular weight fraction of cell-free plasma. In one embodiment the CD36-lipoprotein complex comprises oxidized forms of lipoproteins. For example, sCD36 is associated with oxidized LDL (oxLDL), oxidized LDL, oxidized IDL and/or oxidized VLDL. In yet another embodiment sCD36 is found in complex with oxidized HDL.

In one embodiment the antibody is selected from the group consisting of sc7309 (CD36 (SMf), mouse IgM), and polyclonal CD36 specific antibodies, such as sc5522 (CD36 (N-15), goat IgG, epitope N-terminus (h)), sc9154 (CD36(H-300), rabbit IgG, epitope 1-300 (h)), such as sc5522 and sc9154 (both from Santa Cruz Biotechnology Inc., Santa Cruz, Calif., USA).

The antibodies used in the ELISA assay for detection of circulating CD36 in plasma are preferably labelled for ease of detection, the label preferably being in the form of a biotinylation which is well known to a person skilled in the art.

In particular the CD36-specific monoclonal antibody may be selected from the group consisting of:

### 185-1G2 Vilella

### 131.1 Tandon, Rockville

### 131.2 Tandon, Rockville

### 131.4 Tandon, Rockville

### 131.5 Tandon, Rockville

### 131.7 Tandon, Rockville

### NAM28-8C12 Blanchard, Nantes

### AmAK-5 Kehrel, Muenster

### CLB-IVC7 CLB, Amsterdam

### Lyp 10.5 McGregor, Lyon

### Lyp 13.10 McGregor, Lyon

### Standard Level Used in Diagnosis, Classification and Monitoring

In an individual suffering from an atherosclerotic plaque an increase in the concentration of CD36 polypeptide and/or CD36 encoding nucleic acid molecule of at least 1.15 times of the standard level is an indication of progression of the atherosclerotic plaque towards a symptomatic atherosclerotic plaque. In particular an increase of at least 1.25 of the standard level, for example an increase of at least 1.30, such as increase of at least 1.35 of the standard level, for example 1.40 of the standard level, such as increase of at least 1.45 of the standard level, for example 1.50 of the standard level, such as an increase of at least 1.55 of the standard level, such as an increase of at least 1.60 of the standard level, such as an increase of at least 1.65 of the standard level, such as an increase of at least 1.70 of the standard level, such as increase of at least 1.75 of the standard level, such as an increase of at least 1.80 of the standard level, such as an increase of at least 1.85 of the standard level, such as an increase of at least 2.0 of the standard level, such as an increase of at least 2.25 of the standard level, such as an increase of at least 2.50 of the standard level, such as an increase of at least 3 of the standard level, such as an increase of at least 3.50 of the standard level, such as an increase of at least 4 of the standard level, such as an increase of at least 4.50 of the standard level, such as an increase of at least 5 of the standard level, such as an increase of at least 6, 7, 8, 9 or 10 of the standard level is indicative of progression of the atherosclerotic plaque towards a symptomatic atherosclerotic plaque.

By the term “standard level” is meant the concentration of CD36 polypeptide or CD36 encoding nucleic acid molecule in a pool of samples from a random group of individuals. In one embodiment the standard level is determined in a pool of sample from a random group of asymptomatic individuals. The pool comprises samples from at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or such as at least 100 individuals.

In another embodiment the standard level is the concentration of CD36 polypeptide or CD36 encoding nucleic acid molecule in a pool of samples from a group of individuals such as at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or such as at least 100 individuals, in which no symptoms related to atherosclerosis have been observed more than 2 months ago, more than 3 months ago, more than 4 months ago, more than 5 months ago, or more than 6 months ago at the time for supplying a sample. In one embodiment the standard level is the concentration of CD36 polypeptide or CD36 encoding nucleic acid molecule in a pool of samples from a group of individuals such as at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or such as at least 100 individuals, in which no symptoms related to carotid stenosis and/or carotid sclerosis have been observed more than 2 months ago, more than 3 months ago, more than 4 months ago, more than 5 months ago, or more than 6 months ago at the time for supplying a sample.

### Determination of Treatment Regime

One aspect of the present invention relates to the use of the concentration of CD36 polypeptide or CD36 encoding nucleic acid in respect to determining the treatment regime of an individual based on the concentration of CD36 polypeptide or CD36 encoding nucleic acid.

Thus, the present invention pertains to a method for determining the treatment regime of an individual, said method comprising in a sample from said individual i) determining the concentration of a CD36 polypeptide or part thereof and/or

ii) determining the concentration of a CD36 encoding nucleic acid molecule or part thereof, iii) correlating said concentration determined in i) and/or ii) to a standard level, and iv) based on said correlation according to iii) diagnosing the degree of stenosis caused by atherosclerotic plaques and/or diagnosing the risk of having and/or acquiring a symptomatic atherosclerotic plaque in an individual v) deciding on the treatment regime of said individual based on the outcome of iv).

If it is established based on the methods herein that the individual is at risk of having an increased degree of stenosis the treatment regime of said individual could be determined. For example treatment of the observed stenosis may involve mechanical widening of the arteries through angioplasty and/or stenting of at least one artery of the body, or two, three or four arteries of the body, in a particular embodiment at least one or both of the carotid and/or coronary arteries, respectively, are widened mechanically. Alternatively, the treatment regime may involve endarterectomy involving removal of atheromatous plaque material, or blockage, in the lining of an artery. Endarterectomy may be performed on at least one or both of the carotid and/or coronary arteries, respectively.

Furthermore, if it is established that the individual is at risk of having and/or acquiring a symptomatic atherosclerotic plaque a prophylactic treatment of the individual could be initiated in order to slow and/or inhibit the progression from asymptomatic plaques to symptomatic plaques. For example the treatment regime involves treatment using cholesterol-lowering drugs such as statins. Statins include lovastatin (Mevacor), pravastatin (Pravachol), simvastatin (Zocor), atorvastatin (Lipitor), rosuvastatin (Crestor). Statin drugs are HMG-CoA reductase inhibitors. Without being bound by theory it is believed that statins by inhibiting the HMG-CoA reductase enzyme help to prevent the production of cholesterol within the body. Also combination of statins, niacin and/or intestinal cholesterol absorption-inhibiting supplements (such as ezetimibe) may be used to slow and/or inhibit the progression from asymptomatic plaques to symptomatic plaques. In a further embodiment the treatment regime involves treatment using agents that slow and/or inhibit clotting (the process in which which blood forms solid clots).

### Kit

In one aspect the invention relates to a kit for classifying atherosclerotic plaques in an individual, said method comprising in a sample from said individual, i) determining the concentration of a CD36 polypeptide or part thereof and/or ii) determining the concentration of a CD36 encoding nucleic acid molecule or part thereof, iii) correlating said concentration determined in i) and/or ii) to a standard level, and iv) based on said correlation according to iii) classifying said atherosclerotic plaque.

In another aspect the invention relates to a kit for diagnosing atherosclerotic plaques in an individual, said method comprising in a sample from said individual

i) determining the concentration of a CD36 polypeptide or part thereof and/or ii) determining the concentration of a CD36 encoding nucleic acid molecule or part thereof, iii) correlating said concentration determined in i) and/or ii) to a standard level, and iv) based on said correlation according to iii) diagnosing said atherosclerotic plaques.

In yet another aspect the invention relates to a kit for monitoring an atherosclerotic plaque(s) in an individual, said method comprising in a sample from said individual i) determining the concentration of a CD36 polypeptide or part thereof and/or ii) determining the concentration of a CD36 encoding nucleic acid molecule or part thereof, iii) correlating said concentration determined in i) and/or ii) to a concentration of a CD36 polypeptide or part thereof and/or the concentration of a CD36 encoding nucleic acid molecule or part thereof, measured in the same individual previously and/or iv) correlating said concentration determined in i) and/or ii) to a standard level, and v) based on said correlation according to iii) and/or iv) monitoring any progress of said atherosclerotic plaques.

In a further aspect the invention relates to a kit for diagnosing an individual at risk of having and/or acquiring a symptomatic atherosclerotic plaques, said method comprising in a sample from said individual, i) determining the concentration of a CD36 polypeptide or part thereof and/or ii) determining the concentration of a CD36 encoding nucleic acid molecule or part thereof, iii) correlating said concentration determined in i) and/or ii) to a standard level, and iv) based on said correlation according to iii) diagnosing whether said individual is at risk of having and/or acquiring a symptomatic atherosclerotic plaque.

In yet a further aspect the invention relates to a kit for diagnosing burden of atherosclerotic plaques in an individual, said method comprising in a sample from said individual i) determining the concentration of a CD36 polypeptide or part thereof and/or ii) determining the concentration of a CD36 encoding nucleic acid molecule or part thereof, iii) correlating said concentration determined in i) and/or ii) to a standard level, and iv) based on said correlation according to iii) diagnosing the burden of atherosclerotic plaques in said individual.

Furthermore, the present invention relates to a kit for diagnosing stenosis caused by atherosclerotic plaques in an individual, said method comprising in a sample from said individual i) determining the concentration of a CD36 polypeptide or part thereof and/or ii) determining the concentration of a CD36 encoding nucleic acid molecule or part thereof, iii) correlating said concentration determined in i) and/or ii) to a standard level, and iv) based on said correlation according to iii) diagnosing the degree of stenosis caused by atherosclerotic plaques in an individual.

In addition, the present invention relates to a kit for determining the treatment regime of an individual, said method comprising in a sample from said individual, i) determining the concentration of a CD36 polypeptide or part thereof and/or ii) determining the concentration of a CD36 encoding nucleic acid molecule or part thereof, iii) correlating said concentration determined in i) and/or ii) to a standard level, and iv) based on said correlation according to iii) diagnosing the degree of stenosis caused by atherosclerotic plaques and/or diagnosing the risk of having and/or acquiring a symptomatic atherosclerotic plaque in an individual v) deciding on the treatment regime of said individual based on the outcome of iv).

The one or more kits of the present invention thus comprises at least one detection member for determination of the concentration of a CD36 polypeptide or part thereof and/or for determination of the concentration of a CD36 encoding nucleic acid or part thereof, in particular a transcript.

The detection member is selected from the group of at least one primers, at least one probe and at least one primer pair capable of detecting a CD36 encoding nucleic acid molecule or part thereof as described elsewhere herein.

As described el above the detection member may be any nucleotide probe, such as a DNA, RNA, PNA, or LNA probe capable of hybridising to mRNA indicative of the concentration of CD36 encoding nucleic acid molecule or part thereof, a transcript in the form of a mRNA. The hybridisation conditions are preferably as described herein for probes. In another embodiment the detection member is an antibody capable of specifically binding the CD36 polypeptide or part thereof.

The concentration of a CD36 encoding nucleic acid molecule, a transcript in the form of a mRNA and/or the concentration of a CD36 polypeptide or fragment thereof can be compared manually by a person or by a computer or other machine. An algorithm can be used to detect similarities and differences. The algorithm may score and compare, for example, the genes which are expressed and the genes which are not expressed. Alternatively, the algorithm may look for changes in intensity of expression of a particular gene, transcript or translational product thereof and score changes in intensity between two samples. Similarities may be determined on the basis of genes, transcripts or translational products thereof which are expressed in both samples and genes which are not expressed in both samples or on the basis of genes whose intensity of expression are numerically similar.

Generally, the detection operation will be performed using a reader device external to the diagnostic device. However, it may be desirable in some cases to incorporate the data gathering operation into the diagnostic device itself.

The detection apparatus may be a fluorescence detector, or a spectroscopic detector, or another detector.

Although hybridization is one type of specific interaction which is clearly useful for use in this mapping embodiment antibody reagents may also be very useful.

Gathering data from the various analysis operations, e.g. oligonucleotide and/or microcapillary arrays will typically be carried out using methods known in the art. For example, the arrays may be scanned using lasers to excite fluorescently labeled targets that have hybridized to regions of probe arrays mentioned above, which can then be imaged using charged coupled devices (“CCDs”) for a wide field scanning of the array. Alternatively, another particularly useful method for gathering data from the arrays is through the use of laser confocal microscopy which combines the ease and speed of a readily automated process with high resolution detection.

Following the data gathering operation, the data will typically be reported to a data analysis operation. To facilitate the sample analysis operation, the data obtained by the reader from the device will typically be analyzed using a digital computer. Typically, the computer will be appropriately programmed for receipt and storage of the data from the device, as well as for analysis and reporting of the data gathered, i.e., interpreting fluorescence data to determine the sequence of hybridizing probes, normalization of background and single base mismatch hybridizations, ordering of sequence data in SBH applications, and the like.

### EXPERIMENTALS AND EXAMPLES

### Patients

Carotid plaques from consecutive endarterectomy patients were classified into two groups depending on whether or not the patients had experienced ipsilateral stroke, TIA, or amaurosis fugax in the prior six month to surgery. Plaques were characterized as symptomatic or asymptomatic according to the presence or absence of cerebrovascular symptoms, respectively. The carotid stenoses were diagnosed and classified by precerebral color Duplex ultrasound (Grant E G, Benson C B, Moneta G L, Alexandrov A V, Baker J D, Bluth E I, Carroll B A, Eliasziw M, Gocke J, Hertzberg B S, Katanick S, Needleman L, Pellerito J, Polak J F, Rholl K S, Wooster D L, Zierler R E. Carotid artery stenosis: gray-scale and Doppler US diagnosis—Society of Radiologists in Ultrasound Consensus Conference. Radiology. 2003; 229:340-346) and CT angiography (Anderson G B, Ashforth R, Steinke D E, Ferdinandy R, Findlay J M. CT angiography for the detection and characterization of carotid artery bifurcation disease. Stroke. 2000; 31:2168-2174) according to consensus criteria. The asymptomatic carotid stenoses were detected during clinical examinations of patients with coronary artery disease (CAD), peripheral artery disease, or stroke/TIA more than six month ago.

### Carotid Endarterectomy Specimens

Atherosclerotic carotid plaques were retrieved from patients during carotid endarterectomy. Plaques that were used for protein and RNA extraction were rapidly frozen in liquid nitrogen. For Western blots, the tissue powders from the plaques were homogenized in ice-cold lysis buffer (PBS containing protease inhibitor cocktail [Gibco, Paisley, UK] with 1% Triton X-100 and 0.1% Tween 20) at a ratio of 0.1 ml per 10 mg wet weight tissue by a metal blade homogenizer. Extracts were incubated on ice for 15 minutes, and centrifuged at 12 000 g (15 minutes at 4° C.). The supernatants were retained and protein concentrations in the samples were measured with the BCA method (Pierce, Cheshire, UK).

### Blood Sampling Protocol

Peripheral venous blood was drawn into pyrogen-free EDTA tubes that were immediately immersed in melting ice and centrifuged at 2,500 g for 20 minutes within 20 minutes to obtain platelet-poor plasma. All samples were stored at −80° C. and thawed only once before start of assay procedures.

### High-Density Oligonucleotide Microarrays

Total RNA was isolated from frozen carotid tissue using MagNA Pure Kit III (Roche Applied Science, Indianapolis, Ind.), quantified spectrophotometrically, and stored at −80° C. The Human Genome U133A 2.0 Array encoding 14500 genes was purchased from Affymetrix (Santa Clara, Calif.), and hybridization was performed according to the manufacturer's two cycle target labelling protocol. Briefly, cDNA was prepared from 100 ng total RNA and cRNA was obtained from in vitro transcription of the cDNA. Then the cycle was repeated making cDNA from 600 ng cRNA. Thereafter biotin-labeled cRNA was generated from in vitro transcription of cDNA and fragmented before hybridization to the array. For data analyses, GeneChip Operation Software (1.3) and ArrayAssist (3.4) were used.

### Example 1

### Example of ELISA Analysis Kit for Detecting CD36 in Serum

ELISA analysis, reagents used and assay conditions

Phosphate buffer 0.1 mol/l, pH 8.0
Na₂HPO4, 2H₂O, 0.1 mol/l, pH 8.0, stored at 4° C.

### POD Buffer

NaH₂PO4, H₂O, 1.5 mmol/l
Na₂HPO4, 2H₂O, 8.5 mmol/l
NaCl 400 mmol/l
pH 7.4

### Stored at 4° C. 20

### Preparation of Lysozyme Solution:

Lysozyme, Sigma L-6876, 20 mg/ml. Stored at −20° C. in 1 ml portions, storage life 1 year. Storage life at 4° C. is 4 days.

### POD-Avidin Dako P364: Colour Reagent

TMB Microwell Peroxidase Substrate (1 component)
Cat. No. 50-76-06, Kirkegård and Perry Laboratories. Storage life 1 year at 2-25° C. 25 Phosphoric acid, 1 mol/l

### POD-Avidin Solution:

POD buffer 12 ml
Lysozyme solution 120 μl 30

### POD-avidin 6 μl

To be prepared immediately before use.

### Antibody

Anti-CD36: biotinylated sc-9154 (rabbit polyclonal antibody from Santa Cruz)

### Apparatus

Automatic microtiter plate-rinser Elx50 (Biotek Instruments).

ELISA plates were coated overnight at 4° C. with catcher antibody goat (sc5522)-CD36 (0.1 μg/ml), followed by blocking with phosphate-buffered saline (PBS) and 0.1% Tween for 2 days at 4° C. The plates were stored at −20° C. until use. A microtiter plate coated with antibody against CD36 is rinsed 3 times with 340 μl rinsing buffer pr. well using program 12. The last rinse is terminated without aspiration of rinsing buffer, which is first decanted immediately before application of standards (EDTA-plasma pool) and samples (with possible controls), and immediately before addition of reagent.

### Standards

EDTA blood from 100 subjects from the routine blood sampling are centrifuged at 3000 G for 10 min, the upper part of the plasma is pipetted off and pooled. Pool-plasma is frozen in aliquots of 350 μl and dilutions are used as standard curves. Other EDTA plasma pools are used as high and low internal assay controls.

The intra assay coefficient of variation, which was estimated from a run of 76 single determinations of the same sample, was 10%, and estimated from double determinations of the high control on 15 different days was 6%. The inter assay coefficient of variation, estimated from the controls in each run performed, was 16.4%. Runs were only accepted when controls were within the range of +/−1.96×SD (inter assay).

### Method for Elisa Assay

A microtiter plate is rinsed on the Elx50. Standards, controls, samples and dilution buffer are applied in double rows, 100 μl/well. The positions of the applications are noted. The microtiter plate is covered with plastic film and incubated for 60 min. on a shaking table.

The plate is rinsed on the Elx50. 100 μl biotinylated sc-9154 is added per well, covered with plastic film and incubate 60 min on a shaking table. The plate is rinsed on the Elx50.

100 μl POD-avidin solution is added per well. Covered with plastic film and incubate for about 30 min. on a shaking table. The plate is rinsed on the Elx50. In a fume hood 100 μl TMB is added per well. Cover with plastic film, incubate for about 10 min. on a shaking table. The reaction is terminated with 100 μl phosphoric acid per well (in a fume hood). Cover with plastic film until reading.

### Measurement

Read the extinctions at 450 nm and 620 nm on a Multiscan apparatus before 60 min fol-lowing the termination of the reaction.

### Calculation

Cubic spline with linear scale on both axes. Dilutions of EDTA pool are used as standard curve, and results are expressed relative to the EDTA plasma pool.

Elisa dilution buffer: phosphate buffer 10 mmol/l with 0.145 mol/l NaCl, pH 7.4

### Example 2

### CD36 Gene Expression in Atherosclerotic Plaques

TABLE 1
CD36 was differently expressed in plaques obtained
from asymptomatic (n = 4) compared to symptomatic
(n = 4) carotid atherosclerotic patient. 5 out of 5 probe sets on
the Affimetrix (Human Genome U133A 2.0 Array)
detected CD36 (Hs. 120949)

| UniGene | | Gene | * |
|---|---|---|---|
| ID | Gene Name | Symbol | Ratio |
| Hs. 120949 | CD36 antigen (collagen type I | CD36 | 0.38 |
| | receptor throm-bospondin receptor) | | |
| Hs. 120949 | CD36 antigen (collagen type I | CD36 | 0.22 |
| | receptor throm-bospondin receptor) | | |
| Hs. 120949 | CD36 antigen (collagen type I | CD36 | 0.14 |
| | receptor throm-bospondin receptor) | | |
| Hs. 120949 | CD36 antigen (collagen type I | CD36 | 0.14 |
| | receptor throm-bospondin receptor) | | |
| Hs. 120949 | CD36 antigen (collagen type I | CD36 | 0.07 |
| | receptor throm-bospondin receptor) | | |

*asymptomatic versus symptomatic

### Examples 3 to 5

### Patients

Sixty-two consecutive patients with high-grade internal carotid stenoses (≧70%) were treated with carotid endarterectomy (CEA, 53 patients) or carotid angioplasty with stenting (CAS, 9 patients) (Table 2). The study included 19 (31%) women and 43 (69%) men, aged 65.5 years (range 44-83 years). The patients were classified into two groups according to their symptoms. Sixteen (26%) patients had suffered from clinical symptoms such as stroke, transitory ischemic attack (TIA) or amaurosis fugax ipsilateral to the stenotic internal carotid artery within the past 2 months whereas 46 (74%) patients had symptoms more than 2 months ago (n=22) or had never suffered from symptoms as outlined above (n=24) (Table 2). The carotid stenoses were diagnosed and classified using precerebral color Duplex and CT angiography according to consensus criteria (Anderson et al. Stroke. 2000; Grant E G et al. Radiology. 2003; 229:340-346).

The asymptomatic carotid stenoses were detected during clinical examinations of patients with coronary artery disease (CAD), peripheral artery disease or stroke/TIA more than six months ago. The plaques were also divided into two groups, depending on plaque echogenicity on ultrasound examination according to methods described previously (European Carotid Plaque Study, Eur J Vasc Endovasc Surg. 1995; 10: 23-30; Mathiesen E B et al, Circulation. 2001; 103: 2171-2175), and classified as echolucent or echogenic/heterogeneous. Triplex ultrasound of precerebral arteries, clinical neurological examination, and cerebral diffusion-weighted magnetic resonance imaging (MRI) were performed within two days before and the day after the procedures. Patients with concomitant inflammatory diseases such as infections and autoimmune disorders, or liver or kidney disease were excluded from all parts of the study. The protocols were approved by the regional ethics committee and signed informed consent was obtained from all individuals.

TABLE 2
Baseline variables in patients according to # clinical
symptoms and months after symptoms (n = 62)

| | | | >2 months or | | |
|---|---|---|---|---|---|
| | ≦2 months | | no symptoms | | |
| | (n = 16) | | (n = 46) | | p |
| Age, year | 66 | (7.8) | 65 | (9.3) | .98 |
| Male sex* | 12 | (75) | 31 | (67) | .57 |
| Degree of stenoses, % | 80 | (80-95) | 80 | (70-95) | .65 |
| Echolucent carotid plaque* | 7 | (43.8) | 13 | (28.3) | .25 |
| Ischemia on cerebral MRI* (n = 53) | 11 | (68.8) | 31 | (67.4) | .94 |
| Body mass index, kg/m² | 26.7 | (20.4-35.5) | 25.8 | (19-35.5) | .72 |
| CEA treated* | 15 | (93.8) | 38 | (82.6) | .28 |
| Systolic blood pressure, mmHg | 151 | (110-196) | 150 | (110-194) | .60 |
| Diastolic blood pressure, mmHg | 73 | (32-99) | 80 | (49-101) | .59 |
| Diabetes mellitus* | 4 | (25) | 10 | (22) | .79 |
| Statin treatment* | 13 | (81.3) | 41 | (89.1) | .42 |
| Current smoking* | 7 | (43.8) | 25 | (54.3) | .74 |
| Neopterin, nmol/l | 8.7 | (4.9-18.0) | 9.1 | (5.7-60.3) | .48 |
| CRP, mg/l | 3.5 | (1.0-39.0) | 5 | (0.6-28.0) | .93 |
| Total leukocyte count, 10⁹/l | 8.3 | (4.0-12.1) | 7.5 | (3.8-11.1) | .31 |
| Fibrinogen, g/l | 4.0 | (2.9-6.6) | 3.9 | (2.6-6.9) | .87 |
| Cholesterol, mmol/l | 4.3 | (3.6-5.9) | 4.4 | (2.8-7.5) | .68 |
| HDL cholesterol, mmol/l | 1.3 | (0.7-1.9) | 1.2 | (0.8-2.5) | .82 |
| Triglycerides, mmol/l | 1.4 | (0.6-3.8) | 1.5 | (0.7-3.7) | .31 |
| LDL (n = 49) | 2.6 | (2.0-4.0) | 2.6 | (1.5-5.1) | .46 |
| HbA1c, % | 5.7 | (5.2-6.9) | 5.8 | (0.9-8.8) | .95 |
| β-thromboglobulin, IU/ml | 53.3 | (26.7-142.1) | 54.8 | (15.4-268.5) | .61 |
| Platelet count, 10⁹/l | 225.5 | (132-391) | 261 | (168-441) | .10 |

# Clinical symptoms include stroke, TIA or amaurosis fugax ipsilateral to the stenotic internal carotid artery. Numbers are un-adjusted means (SD) or *numbers (percentages)

### Blood Sampling Protocol

Peripheral venous blood was drawn into pyrogen-free EDTA tubes that were immediately immersed in melting ice and centrifuged at 2,500 g for 25 minutes at 4° C. within 20 minutes to obtain platelet-poor plasma. All samples were stored at −80° C. and thawed only once.

### Immunohistochemistry

Acetone-fixed sections of atherosclerotic carotid plaques, retrieved from patients during carotid endarterectomy, were stained using monoclonal mouse anti-human CD36 (FA6-152, Novus Biologicals, Littleton, Colo.), anti-human CD3 IgG (DakoCytomation, Glostrup, Denmark), and anti-human smooth muscle actin (DakoCytomation), affinity purified polyclonal mouse anti-human macrophages (calprotectin) IgG (MCA874G, Serotec Ltd., Oxford, UK), and sheep anti-rat von Willebrand factor IgG (Cedarlane, Ontario, Canada). The primary antibodies were followed by biotinylated anti-mouse or anti-sheep IgG (Vector Laboratories, Burlingame, Calif.). The immunoreactivities were further amplified using avidin-biotin-peroxidase complexes (Vectastain Elite kit, Vector Laboratories). Diaminobenzidine was used as the chromogen in a commercial metal enhanced system (Pierce Chemical, Rockford, Ill.). The sections were counter-stained with hematoxylin. Omission of the primary antibody served as a negative control.

### Soluble CD36 Enzyme-Linked Immunoassay (ELISA)

ELISA plates were coated overnight at 4° C. with catcher antibody goat (sc5522)-CD36 (0.1 μg/ml), followed by blocking with phosphate-buffered saline (PBS) and 0.1% Tween for 2 days at 4° C. The plates were stored at −20° C. until use. Detection anti-body sc9154 was biotinylated as described elsewhere (Glintborg D et al. Published online ahead of print Nov. 13, 2007). All antibodies were from Santa Cruz Biotechnology (Santa Cruz, Calif.). A pool of EDTA-plasma from 100 subjects from routine blood sampling served as standard plasma, and was applied in increasing dilutions in duplicates. Other pools of EDTA-plasma served as high and low controls and PBS was used as background control. Patient samples were applied in appropriate dilutions in duplicates. Following 1 hour incubation at room temperature, the wells were rinsed, and biotinylated anti CD36 (sc9154) was applied and incubated for 30 minutes. After rinsing, TMB Microwell Peroxidase Substrate (KemEnTec, Copenhagen, Denmark) was added. The reaction was terminated by phosphorous acid, and extinctions were determined at 450 nm and 620 nm on a Multiscan Apparatus (Thermo, Langenselbold, Germany). Absorptions were calculated relative to the standard EDTA plasma pool and expressed as relative units. Controls were allowed to deviate by 1 SD from the true value. Intra-assay and long-term inter-assay coefficient of variation were 6% and 16%, respectively.

### Miscellaneous

C-reactive protein (CRP) levels were determined by a high-sensitivity particle enhanced immunoturbidimetric assay on a modular platform (Roche Diagnostic, Basel, Switzerland). Concentrations of lipid parameters and HbA1c were measured by routine laboratory methods as previously described. Plasma levels of neopterin and β-thromboglobulin (□-TG) were measured by ELISAs obtained from IBL Hamburg (Hamburg, Germany) and Diagnostica Stago (Asnieres, France), respectively.

### Statistical Analyses

For comparisons of 2 groups of individuals, the Mann-Whitney U test was used. When comparing 3 groups of individuals, the non-parametric Kruskal-Wallis test was used. If a significant difference was found, Mann-Whitney U test was used to calculate the difference between each pair of groups. Coefficients of correlation (r) were calculated by the Spearman rank test. The Fisher's exact test was used when comparing proportions. The relationship between variables was calculated using the linear and binary regression analysis for continuous and categorical dependent variables, respectively. Probability values (2-sided) were considered significant at value of <0.05.

### Example 3

### Plasma CD36 and Plaque Stability

In contrast to CRP and neopterin, plasma levels of sCD36 were significantly higher in those who had symptoms related to their carotid stenosis within the last two months compared with the other patients (2.17 [0.58-4.74] versus 1.27[0-6.03], p=0.019, data are given in relative units as medians and ranges). Moreover, when patients were divided into three groups according to their latest clinical symptoms (i.e., symptoms within the last 2 months [n=16], symptoms within the last 3-6 months [n=15], or asymptomatic plaques, i.e., no symptoms or symptoms more than 6 months ago [n=31]), the former group had significantly raised plasma levels of sCD36 as compared with the other two groups (FIG. 2). Within the patient group as a whole, plasma levels of sCD36 were significantly correlated with total leukocyte counts (r=0.43, p<0.02) and fibrinogen (r=−0.40, p<0.02). However, after adjustment for these parameters, sCD36 was still a significant predictor of clinical symptoms from the plaques within the last 2 months (p=0.049, CI: 1.0-2.7). We have previously reported raised plasma levels of sCD36 in type 2 diabetes and in obese individuals, but in the present study we found no relationship between sCD36 and either BMI, the occurrence of diabetes mellitus or glycemic control (i.e., HbA1c) (data not shown).

We have previously demonstrated that CD36 exists in a soluble form in plasma, with elevated levels in patients with type 2 diabetes and in patients with the polycystic ovary syndrome in relation to risk factors for atherosclerosis such as insulin resistance and glycemic control (Handberg A et al. Circulation. 2006; 114:1169-1176; Glintborg D et al. Diabetes Care. Published online ahead of print Nov. 13, 2007. In the present study, we extended these findings by showing a more direct relationship between plasma levels of sCD36 and the nature of the atherosclerotic lesion, independent of BMI and the occurrence of diabetes. Thus, while there were no differences in traditional risk markers for cardiovascular disease or established markers of systemic inflammation and platelet activation between those with symptomatic disease in carotid arteries within the preceding 2 months and the other patients with carotid atherosclerosis, plasma levels of sCD36 were significantly raised in the former group of patients. Without being bound by theory one possible cellular source of sCD36 in these patients, based on our previous observation of increased CD36 mRNA levels in symptomatic carotid plaques combined with our present data showing strong immunostaining of CD36 in macrophages within symptomatic as compared asymptomatic plaques, may suggest that the raised plasma levels of sCD36 could reflect increased release from the symptomatic and unstable lesion.

### Example 4

### Plasma CD36 and Plaque Morphology

Ultrasound plaque appearance or echogenicity can in principle be classified into plaques with low-level echoes, with a thin often incomplete shell on the luminal surface (echolucent plaque), and plaques with medium and high level echoes often reflecting a higher content of dense fibrous tissue and calcification (echogenic/heterogeneous plaques). Data on plaque morphology were available in 59 of the patients, and as can be seen in FIG. 4, patients with echolucent plaques (n=20) tended to have higher sCD36 levels compared with those with echogenic/heterogeneous plaques (n=39, p=0.09).

### Example 5

### CD36 Expression within the Carotid Lesion

The cellular source of plasma levels of sCD36 in patients with symptomatic carotid lesions is not clear, but we have recently identified CD36 as one of 87 genes that was markedly up-regulated in symptomatic carotid plaques, suggesting that plaque in itself could contribute to the raised plasma levels of sCD36 in these patients. To further elucidate these issues, immunohistochemical analysis was performed on carotid plaques from 2 patients with symptomatic disease (<2 months) and 2 patients with asymptomatic disease (FIG. 6). Staining of serial sections of these atherosclerotic lesions showed anti-CD36 immunostaining in plaques from symptomatic patients that were localized to the lipid-rich core of the plaque (FIG. 6A) with strong immunostaining against calprotectin-positive macrophages (FIG. 6B). Different intensity of CD36 immunoreactivity was seen in the core region with regions with very weak or no anti-CD36 immunostaining, as in the acellular center of the core, while other regions displayed more intense immunostaining. As demonstrated in FIG. 6, regions of the plaque adjacent to the media displayed the strongest CD36 immunoreactivity. However, no anti-CD36 immunostaining was seen outside the core. CD36 immunostaining was also seen in lipid-rich calprotectin-positive regions in lesions from patients with asymptomatic disease, but in these patients, the atherosclerotic plaques were less advanced and the region with CD36-positive immunostaining was smaller than in lesions from patients with symptomatic disease.

CD36 is believed to play a critical role in the initiation and progression of atherosclerotic lesions through its ability to bind and internalize modified LDL trapped in the arterial wall, facilitating the formation of lipid-engorged macrophage foam cells (Collot-Teixeira S et al. Cardiovasc Res. 2007; 75:468-477; Tuomisto T T et al. Atherosclerosis. 2005; 180:283-291. Indeed, previous studies have reported accelerated CD36 expression in parallel with the progression of atherosclerosis 6, especially located to foamed, large-sized macrophages (Nakata Aet al. Arterioscler Thromb Vasc Biol. 1999; 19:1333-1339. Our finding in the present study of strong immunostaining of CD36 in symptomatic as compared with asymptomatic plaques, primarily located to lipid-loaded macrophages in the fatty core of the atherosclerotic plaque, further support a relationship between enhanced CD36 and advanced atherosclerosis. Furthermore, plasma levels of sCD36 were markedly elevated in those with symptoms related to their carotid stenosis within the last two months as compared with other patients. The mechanisms for release of CD36 in its soluble form are at present unclear, but it has been suggested that plasma levels of sCD36 could serve as a biomarker of conditions with altered CD36 expression such as elevated levels of modified lipoproteins and low-grade inflammation (Tuomisto T T et al. Atherosclerosis. 2005; 180:283-291).

Moreover, foam cell apoptosis has been linked to plaque destabilization and thrombus formation with subsequent development of acute ischemic events (Littlewood T D and Bennett. Curr OpinLipidol. 2003; 14:469-475), and it may be speculated that apoptosis of lipid-loaded macrophages may lead to enhanced release of CD36. It is therefore tempting to hypothesize that the increased plasma levels of sCD36 in patients with recent symptomatic carotid plaques, with a time-dependent relationship with the acute symptoms, at least partly reflects intensified release of sCD36 during plaque destabilization.

CD36 is expressed not only on monocytes and macrophages, but also on endothelial cells Adachi H and Tsujimoto M. Prog Lipid Res. 2006; 45:379-404 and platelets (Ikeda H. Hokkaido Igaku Zasshi. 1999; 74:99-104. Recently, Podrez and co-workers proposed that platelet-related CD36 could be a sensor of oxidative stress and a modulator of platelet activation, promoting thrombosis under hyperlipidemic conditions (Podrez E A et al. Nat. Med. 2007; 13:1086-1095) such as during interaction between platelets and an unstable atherosclerotic lesion. In contrast to sCD36, we found no relationship between plasma levels of CRP, neopterin, and beta-TG, which are reliable markers of systemic inflammation and monocyte/macrophage and platelet activation, respectively. It is possible that the ability of sCD36 to reflect plaque instability and symptomatic disease may reflect its capacity to mirror several pathogenic processes involved in plaque destabilization, such as activation of platelets and monocytes/macrophages, as well as pathogenic events within the lesion such as foam cell apoptosis and interactions between platelets and the atherosclerotic plaque. We propose that sCD36 in plasma is a marker of plaque instability and symptomatic carotid atherosclerosis, possibly at least partly as a result of CD36 release into the circulation from the foam cell infiltrated layer overlaying the fatty core, and that this release is intensified during plaque rupture. The results of the present study suggest sCD36 to predict forthcoming cardiovascular events.

### REFERENCES

All literature and patent references herein are incorporated by reference including the following selected references on CD36:
Anderson G B, Ashforth R, Steinke D E, Ferdinandy R, Findlay J M. CT Angiography for the detection and characterization of carotid artery bifurcation disease, Stroke. 2000; 31:2168-2174
Grant E G, Benson C B, Moneta G L, Alexandrov A V, Baker J D, Bluth E I, Carroll B A, Eliasziw M, Gocke J, Hertzberg B S, Katarick S, Needleman L, Pellerito J, Polak J F, Rholl K S, Wooster D L, Zierler E. Carotid artery stenosis: gray-scale and doppler US diagnosis—Society of Radiologists in Ultrasound Consensus Conference, Radiology. 2003; 229:340-346
European Carotid Plaque Study G, Carotid artery plaque composition relationship to clinical presentation and ultrasound B-mode imaging, Eur J Vasc Endovasc Surg. 1995; 10: 23-30
Mathiesen E B, Bonaa K H, Joakimsen O. Echolucent plaques are associated with high risk of ischemic cerebrovascular events in carotid stenosis: the Tromsø study, Circulation. 2001; 103: 2171-2175
Handberg A, Levin K, Højlund K, Beck-Nielsen H. Identification of the oxidized low-density lipoprotein scavenger receptor CD36 in plasma: A novel marker of insulin resistance. Circulation. 2006; 114:1169-1176
Glintborg D, Højlund K, Andersen M, Henriksen J E, Beck-Nielsen H, Handberg A. Soluble CD36 and risk markers of insulin resistance and atherosclerosis are elevated in polycystic ovary syndrome and significantly reduced during pioglitazone treatment. Diabetes Care. Published online ahead of print Nov. 13, 2007.
Collot-Teixeira S, Martin J, McDermott-Roe C, Poston R, McGregor J L. CD36 and macrophages in atherosclerosis. Cardiovasc Res. 2007; 75:468-477
Tuomisto T T, Riekkinen M S, Viita H, Levonen A L, Yla-Herttuala S. Analysis of gene and protein expression during monocyte-macrophage differentiation and cholesterol loading-cDNA and protein array study. Atherosclerosis. 2005; 180:283-291.
Nakata A, Nakagawa Y, Nishida M, Nozaki S, Miyagawa J, Nakagawa T, Tamura R, Matsumoto K, Kameda-Takemura K, Yamashita S, Matsuzawa Y. CD36, a novel receptor for oxidized low-density lipoproteins, is highly expressed on lipidladen macrophages in human atherosclerotic aorta. Arterioscler Thromb Vasc Biol. 1999; 19:1333-1339.
Littlewood T D, Bennett M R. Apoptotic cell death in atherosclerosis. Curr Opin Lipidol. 2003; 14:469-475.
Adachi H, Tsujimoto M. Endothelial scavenger receptors. Prog Lipid Res. 2006; 45:379-404.
Ikeda H. Platelet membrane protein CD36. Hokkaido Igaku Zasshi. 1999; 74:99-104.
Podrez E A, Byzova T V, Febbraio M, Salomon R G, Ma Y, Valiyaveettil M, Poliakov E, Sun M, Finton P J, Curtis B R, Chen J, Zhang R, Silverstein R L, Hazen S L. Platelet CD36 links hyperlipidemia, oxidant stress and a prothrombotic phenotype. Nat. Med. 2007; 13:1086-1095

1. A method for classifying atherosclerotic plaques as unstable in an individual, said method comprising:
i) determining the concentration of a CD36 polypeptide or part thereof in a body fluid sample from said individual; and/or
ii) determining the concentration of a CD36 encoding nucleic acid molecule or part thereof in a body fluid sample from said individual;
iii) correlating said concentration determined in i) and/or ii) to a standard level of CD36; and
iv) based on said correlation according to iii) classifying said atherosclerotic plaques as unstable.

2. A method for diagnosing atherosclerotic plaques as unstable in an individual, said method comprising:
i) determining the concentration of a CD36 polypeptide or part thereof in a body fluid sample from said individual; and/or
ii) determining the concentration of a CD36 encoding nucleic acid molecule or part thereof in a body fluid sample from said individual;
iii) correlating said concentration determined in i) and/or ii) to a standard level of CD36; and
iv) based on said correlation according to iii) diagnosing said atherosclerotic plaques as unstable.

3. A method for monitoring progress of atherosclerotic plaques in an individual, said method comprising:
i) determining the concentration of a CD36 polypeptide or part thereof in a body fluid sample from said individual, and/or
ii) determining the concentration of a CD36 encoding nucleic acid molecule or part thereof in a body fluid sample from said individual;
iii) correlating said concentration determined in i) and/or ii) to a concentration of a CD36 polypeptide or part thereof and/or the concentration of a CD36 encoding nucleic acid molecule or part thereof, measured in the same individual previously, and/or
iv) correlating said concentration determined in i) and/or ii) to a standard level of CD36; and
v) based on said correlation according to iii) and/or iv) monitoring any progress of said atherosclerotic plaques.

4. A method for diagnosing an individual at risk of having and/or acquiring unstable atherosclerotic plaques, said method comprising:
i) determining the concentration of a CD36 polypeptide or part thereof in a body fluid sample from said individual; and/or
ii) determining the concentration of a CD36 encoding nucleic acid molecule or part thereof in a body fluid sample from said individual;
iii) correlating said concentration determined in i) and/or ii) to a standard level of CD36; and
iv) based on said correlation according to iii) diagnosing whether said individual is at risk of having and/or acquiring unstable atherosclerotic plaques.

5. A method for diagnosing a burden of unstable atherosclerotic plaques in an individual, said method comprising:
i) determining the concentration of a CD36 polypeptide or part thereof in a body fluid sample from said individual; and/or
ii) determining the concentration of a CD36 encoding nucleic acid molecule or part thereof in a body fluid sample from said individual;
iii) correlating said concentration determined in i) and/or ii) to a standard level of CD36; and
iv) based on said correlation according to iii) diagnosing the burden of unstable atherosclerotic plaques in said individual.

6. A method for diagnosing stenosis caused by unstable atherosclerotic plaques in an individual, said method comprising:
i) determining the concentration of a CD36 polypeptide or part thereof in a body fluid sample from said individual; and/or
ii) determining the concentration of a CD36 encoding nucleic acid molecule or part thereof in a body fluid sample from said individual;
iii) correlating said concentration determined in i) and/or ii) to a standard level of CD36; and
iv) based on said correlation according to iii) diagnosing a degree of stenosis caused by unstable atherosclerotic plaques in said individual.

7. A method for determining a treatment regime of an individual, said method comprising:
i) determining the concentration of a CD36 polypeptide or part thereof in a body fluid sample from said individual; and/or
ii) determining the concentration of a CD36 encoding nucleic acid molecule or part thereof in a body fluid sample from said individual;
iii) correlating said concentration determined in i) and/or ii) to a standard level of CD36;
iv) based on said correlation according to iii) diagnosing a degree of stenosis caused by unstable atherosclerotic plaques, and/or diagnosing a risk of having and/or acquiring unstable atherosclerotic plaques in said individual; and
v) deciding on a treatment regime of said individual based on the diagnosis of iv).

8. (canceled)

9. The method according to claim 1, wherein said sample is a cell-free sample.

10. The method according to claim 1, wherein said sample is a plasma sample.

11-12. (canceled)

13. The method according to claim 1, wherein an increase of the CD36 concentration to at least 1.15 times the standard level is an indication of an atherosclerotic plaque in progression.

14. The method according to claim 1, wherein said concentration of said CD36 polypeptide or part thereof is determined by detection of:
i) an amino acid sequence consisting of SEQ ID NO: 4, SEQ ID NO: 5, SEQ ID NO: 6, SEQ ID NO: 7, SEQ ID NO: 8 and/or SEQ ID NO: 9,
ii) an amino acid sequence having at least 90% sequence identity with a sequence of (i), or with a fragment thereof, and/or
iii) an amino acid sequence complementary to any of the sequences of (i) or (ii).

15. The method according to claim 1, wherein said amount of a CD36 encoding nucleic acid molecule or part thereof is determined by detection of:
i) a nucleotide sequence consisting of SEQ ID NO: 1 SEQ ID NO: 2 and/or SEQ ID NO: 3,
ii) a nucleotide sequence having at least 90% sequence identity with a sequence of (i), or with a fragment thereof, and/or
iii) a nucleotide sequence complementary to any of the sequences of (i) or (ii).

16-17. (canceled)

18. The method according to claim 1, wherein the CD36 polypeptide concentration is determined by a method comprising:
i) providing a sample to be investigated;
ii) providing an anti-CD36 antibody;
iii) exposing the sample to the anti-CD36 antibody;
iv) optionally, exposing said CD36-antibody complex to at least a further antibody directed against said CD36-antibody complex; and
v) detecting and quantifying the amount of said anti-CD36 antibody of (iii) or optionally said further antibody of (iv).

19. The method according to claim 1, wherein the CD36 polypeptide concentration is determined by a method comprising:
(i) providing a plasma sample to be investigated,
(ii) providing an anti-CD36 antibody,
(iii) exposing the sample to be investigated to the anti-CD36 antibody bound to a solid phase,
(iv) optionally exposing the CD36-antibody complex to a second anti-CD36 antibody, and
(iv) detecting and quantifying the amount of the anti-CD36 antibody which binds to CD36.

20. The method according to claim 19 comprising the use of a solid phase ELISA enzyme immunoassay.

21. The method of claim 19, wherein the solid phase is a microtiter plate.

22. The method of claim 19, wherein said anti-CD36 antibody is selected from the group consisting of monoclonal and polyclonal CD36 specific antibodies.

23. The method of claim 22, wherein said anti-CD36 antibody is selected from the group of antibodies consisting of sc5522 (CD36 (N-15), goat IgG, epitope N-terminus (h)), sc9154 (CD36 (H-300), rabbit IgG, epitope 1-300 (h)), and sc7309 (CD36 (SMf), mouse IgM).

24. A kit for use in the method as defined in claim 1, comprising at least one detection member.

25. The kit according to claim 24, wherein said detection member is at least one antibody, wherein said antibody is directed against CD36 polypeptide or a part thereof.

26. The kit according to claim 24, wherein said detection member is selected from the group consisting of at least one primer, at least one probe, and at least one primer pair capable of detecting a CD36 encoding nucleic acid molecule.


SEQUENCE LISTING (45 sequences)

SEQ ID NO: 1 - DNA, 2338 bp, Homo sapiens
gaggactgcagtgtaggactttcctgcagaataccatttgatcctattaagaattgtccaaatgttggagcatttgattgaaaaatccttcttagccattttaaagatagctttccaatgattagacgaattgattctttctgtgactcatcagttcatttcctgtaaaattcatgtcttgctgttgatttgtgaataagaaccagagcttgtagaaaccactttaatcatatccaggagtttgcaagaaacaggtgcttaacactaattcacctcctgaacaagaaaaatgggctgtgaccggaactgtgggctcatcgctggggctgtcattggtgctgtcctggctgtgtttggaggtattctaatgccagttggagacctgcttatccagaagacaattaaaaagcaagttgtcctcgaagaaggtacaattgcttttaaaaattgggttaaaacaggcacagaagtttacagacagttttggatctttgatgtgcaaaatccacaggaagtgatgatgaacagcagcaacattcaagttaagcaaagaggtccttatacgtacagagttcgttttctagccaaggaaaatgtaacccaggacgctgaggacaacacagtctctttcctgcagcccaatggtgccatcttcgaaccttcactatcagttggaacagaggctgacaacttcacagttctcaatctggctgtggcagctgcatcccatatctatcaaaatcaatttgttcaaatgatcctcaattcacttattaacaagtcaaaatcttctatgttccaagtcagaactttgagagaactgttatggggctatagggatccatttttgagtttggttccgtaccctgttactaccacagttggtctgttttatccttacaacaatactgcagatggagtttataaagttttcaatggaaaagataacataagtaaagttgccataatcgacacatataaaggtaaaaggaatctgtcctattgggaaagtcactgcgacatgattaatggtacagatgcagcctcatttccaccttttgttgagaaaagccaggtattgcagttcttttcttctgatatttgcaggtcaatctatgctgtatttgaatccgacgttaatctgaaaggaatccctgtgtatagatttgttcttccatccaaggcctttgcctctccagttgaaaacccagacaactattgtttctgcacagaaaaaattatctcaaaaaattgtacatcatatggtgtgctagacatcagcaaatgcaaagaagggagacctgtgtacatttcacttcctcattttctgtatgcaagtcctgatgtttcagaacctattgatggattaaacccaaatgaagaagaacataggacatacttggatattgaacctataactggattcactttacaatttgcaaaacggctgcaggtcaacctattggtcaagccatcagaaaaaattcaagtattaaagaatctgaagaggaactatattgtgcctattctttggcttaatgagactgggaccattggtgatgagaaggcaaacatgttcagaagtcaagtaactggaaaaataaacctccttggcctgatagaaatgatcttactcagtgttggtgtggtgatgtttgttgcttttatgatttcatattgtgcatgcagatcgaaaacaataaaataaacctggctcaagcacaaaccaatttgtgttgttctgattcaataattggtttctgggtggccaattcagaagaagagtgtacatgctcaacaaatcctaggccctgcattcctgtcatcctcatccgggggaaacaccatcatcccagtagctgccctattcaactgcaacagtctccaggaccatcagtatactgcatttcatgtgcaccaaatattttgaaagacatttataaataattggcttatgactcatatttctctatgaataccttcatacagcaggtataactcttttctttatgggcttaaatattttgtcactgatcctgcaaatggacatcattttagcacactagcggtttatattttaaggaccttcattctctgttctgcacctcttctggaaattgagtaaattttgcttttttttttttactcagttgcaacttacgcttggcatcttcagaatgcttttctagcattaagagatgtaaatgataaaggaattattgtatgaaatattacaaagcgtagactatgcattgttattcattataatattttttgctgtcataatcgcctcataaagacaggtttcaaccattaaaatatgttcttccttaaaaaa

SEQ ID NO: 2 - DNA, 1983 bp, Homo sapiens
gaggactgcagtgtaggactttcctgcagaataccatttgatcctattaagaattgtccaaatgttggagcatttgattgaaaaatccttcttagccattttaaagatagctttccaatgattagacgaattgattctttctgtgactcatcagttcatttcctgtaaaattcatgtcttgctgttgatttgtgaataagaaccagagcttgtagaaaccactttaatcatatccaggagtttgcaagaaacaggtgcttaacactaattcacctcctgaacaagaaaaatgggctgtgaccggaactgtgggctcatcgctggggctgtcattggtgctgtcctggctgtgtttggaggtattctaatgccagttggagacctgcttatccagaagacaattaaaaagcaagttgtcctcgaagaaggtacaattgcttttaaaaattgggttaaaacaggcacagaagtttacagacagttttggatctttgatgtgcaaaatccacaggaagtgatgatgaacagcagcaacattcaagttaagcaaagaggtccttatacgtacagagttcgttttctagccaaggaaaatgtaacccaggacgctgaggacaacacagtctctttcctgcagcccaatggtgccatcttcgaaccttcactatcagttggaacagaggctgacaacttcacagttctcaatctggctgtggcagctgcatcccatatctatcaaaatcaatttgttcaaatgatcctcaattcacttattaacaagtcaaaatcttctatgttccaagtcagaactttgagagaactgttatggggctatagggatccatttttgagtttggttccgtaccctgttactaccacagttggtctgttttatccttacaacaatactgcagatggagtttataaagttttcaatggaaaagataacataagtaaagttgccataatcgacacatataaaggtaaaaggaatctgtcctattgggaaagtcactgcgacatgattaatggtacagatgcagcctcatttccaccttttgttgagaaaagccaggtattgcagttcttttcttctgatatttgcaggtcaatctatgctgtatttgaatccgacgttaatctgaaaggaatccctgtgtatagatttgttcttccatccaaggcctttgcctctccagttgaaaacccagacaactattgtttctgcacagaaaaaattatctcaaaaaattgtacatcatatggtgtgctagacatcagcaaatgcaaagaagggagacctgtgtacatttcacttcctcattttctgtatgcaagtcctgatgtttcagaacctattgatggattaaacccaaatgaagaagaacataggacatacttggatattgaacctataactggattcactttacaatttgcaaaacggctgcaggtcaacctattggtcaagccatcagaaaaaattcaagtattaaagaatctgaagaggaactatattgtgcctattctttggcttaatgagactgggaccattggtgatgagaaggcaaacatgttcagaagtcaagtaactggaaaaataaacctccttggcctgatagaaatgatcttactcagtgttggtgtggtgatgtttgttgcttttatgatttcatattgtgcatgcagatcgaaaacaataaaataagtaagtatgtaccaaaaaatattgcttcaataatattagcttatatattacttgttttcactttatcaaagagaagttacatattaggccatatatatttctagacatgtctagccactgatcatttttaaatataggtaaataaacctataaatattatcacgcagatcactaaagtatatctttaattctgggagaaatgagataaaagatgtacttgtgaccattgtaacaatagcacaaataaagcacttgtgccaaagttgtccaaaaaa

SEQ ID NO: 3 - DNA, 2050 bp, Homo sapiens
agatgtcaggataaccttaaggatagatgaagggttgagagcctgtgcctcatttctgagttctcagctgctatgccgtggaaatcctgtttactttctgcatctgctcctgcaagactctggagccagtcttgaggtcctacatctccgaaagcaagctcttctagaagttgatagctttccaatgattagacgaattgattctttctgtgactcatcagttcatttcctgtaaaattcatgtcttgctgttgatttgtgaataagaaccagagcttgtagaaaccactttaatcatatccaggagtttgcaagaaacaggtgcttaacactaattcacctcctgaacaagaaaaatgggctgtgaccggaactgtgggctcatcgctggggctgtcattggtgctgtcctggctgtgtttggaggtattctaatgccagttggagacctgcttatccagaagacaattaaaaagcaagttgtcctcgaagaaggtacaattgcttttaaaaattgggttaaaacaggcacagaagtttacagacagttttggatctttgatgtgcaaaatccacaggaagtgatgatgaacagcagcaacattcaagttaagcaaagaggtccttatacgtacagagttcgttttctagccaaggaaaatgtaacccaggacgctgaggacaacacagtctctttcctgcagcccaatggtgccatcttcgaaccttcactatcagttggaacagaggctgacaacttcacagttctcaatctggctgtggcagctgcatcccatatctatcaaaatcaatttgttcaaatgatcctcaattcacttattaacaagtcaaaatcttctatgttccaagtcagaactttgagagaactgttatggggctatagggatccatttttgagtttggttccgtaccctgttactaccacagttggtctgttttatccttacaacaatactgcagatggagtttataaagttttcaatggaaaagataacataagtaaagttgccataatcgacacatataaaggtaaaaggaatctgtcctattgggaaagtcactgcgacatgattaatggtacagatgcagcctcatttccaccttttgttgagaaaagccaggtattgcagttcttttcttctgatatttgcaggtcaatctatgctgtatttgaatccgacgttaatctgaaaggaatccctgtgtatagatttgttcttccatccaaggcctttgcctctccagttgaaaacccagacaactattgtttctgcacagaaaaaattatctcaaaaaattgtacatcatatggtgtgctagacatcagcaaatgcaaagaagggagacctgtgtacatttcacttcctcattttctgtatgcaagtcctgatgtttcagaacctattgatggattaaacccaaatgaagaagaacataggacatacttggatattgaacctataactggattcactttacaatttgcaaaacggctgcaggtcaacctattggtcaagccatcagaaaaaattcaagtattaaagaatctgaagaggaactatattgtgcctattctttggcttaatgagactgggaccattggtgatgagaaggcaaacatgttcagaagtcaagtaactggaaaaataaacctccttggcctgatagaaatgatcttactcagtgttggtgtggtgatgtttgttgcttttatgatttcatattgtgcatgcagatcgaaaacaataaaataagtaagtatgtaccaaaaaatattgcttcaataatattagcttatatattacttgttttcactttatcaaagagaagttacatattaggccatatatatttctagacatgtctagccactgatcatttttaaatataggtaaataaacctataaatattatcacgcagatcactaaagtatatctttaattctgggagaaatgagataaaagatgtacttgtgaccattgtaacaatagcacaaataaagcacttgtgccaaagttgtccaaaaaa

SEQ ID NO: 4 - protein, 472 aa, Homo sapiens
MGCDRNCGLIAGAVIGAVLAVFGGILMPVGDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAAASHIYQNQFVQMILNSLINKSKSSMFQVRTLRELLWGYRDPFLSLVPYPVTTTVGLFYPYNNTADGVYKVFNGKDNISKVAIIDTYKGKRNLSYWESHCDMINGTDAASFPPFVEKSQVLQFFSSDICRSIYAVFESDVNLKGIPVYRFVLPSKAFASPVENPDNYCFCTEKIISKNCTSYGVLDISKCKEGRPVYISLPHFLYASPDVSEPIDGLNPNEEEHRTYLDIEPITGFTLQFAKRLQVNLLVKPSEKIQVLKNLKRNYIVPILWLNETGTIGDEKANMFRSQVTGKINLLGLIEMILLSVGVVMFVAFMISYCACRSKTIK

SEQ ID NO: 5 - protein, 472 aa, Homo sapiens; identical to SEQ ID NO: 4

SEQ ID NO: 6 - protein, 472 aa, Homo sapiens; identical to SEQ ID NO: 4

SEQ ID NO: 7 - protein, 472 aa, Homo sapiens; identical to SEQ ID NO: 4

SEQ ID NO: 8 - protein, 472 aa, Homo sapiens; identical to SEQ ID NO: 4

SEQ ID NO: 9 - protein, 472 aa, Homo sapiens; identical to SEQ ID NO: 4

SEQ ID NO: 10 - DNA, 277 bp, Homo sapiens
aactggattcactttacaatttgcaaaacggctgcaggtcaacctattggtcaagccatcagaaaaaattcaagtattaaagaatctgaagaggaactatattgtgcctattctttggcttaatgagactgggaccattggtgatgagaaggcaaacatgttcagaagtcaagtaactggaaaaataaacctccttggcctgatagaaatgatcttactcagtgttggtgtggtgatgtttgttgcttttatgatttcatattgtgcatgcagatcg

SEQ ID NO: 11 - DNA, 25 bp, Artificial
  Probe
aactggattcactttacaatttgca

SEQ ID NO: 12 - DNA, 25 bp, Artificial
  Probe
gcaaaacggctgcaggtcaacctat

SEQ ID NO: 13 - DNA, 25 bp, Artificial
  Probe
gtcaacctattggtcaagccatcag

SEQ ID NO: 14 - DNA, 25 bp, Artificial
  Probe
ggaactatattgtgcctattctttg

SEQ ID NO: 15 - DNA, 25 bp, Artificial
  Probe
gtgcctattctttggcttaatgaga

SEQ ID NO: 16 - DNA, 25 bp, Artificial
  Probe
aatgagactgggaccattggtgatg

SEQ ID NO: 17 - DNA, 25 bp, Artificial
  Probe
tggaaaaataaacctccttggcctg

SEQ ID NO: 18 - DNA, 25 bp, Artificial
  Probe
cctccttggcctgatagaaatgatc

SEQ ID NO: 19 - DNA, 25 bp, Artificial
  Probe
gatcttactcagtgttggtgtggtg

SEQ ID NO: 20 - DNA, 25 bp, Artificial
  Probe
ttggtgtggtgatgtttgttgcttt

SEQ ID NO: 21 - DNA, 25 bp, Artificial
  Probe
gatttcatattgtgcatgcagatcg

SEQ ID NO: 22 - DNA, 491 bp, Homo sapiens
aaccaatttgtgttgttctgattcaataattggtttctgggtggccaattcagaagaagagtgtacatgctcaacagtctccaggaccatcagtatactgcatttcatgtgcaccaaatattttgaaagacatttataaataattggcttatgactcatatttctctatgaataccttcatacagcaggtataactcttttctttatgggcttaaatattttgtcactgatcctgcaaatggacatcattttagcacactagcggtttatattttaaggaccttcattctctgttctgcacctcttctggaaattgagtaaattttgctttttttttttactcagttgcaacttacgcttggcatcttcagaatgcttttctagcattaagagatgtaaatgataaaggaattattgtatgaaatattacaaagcgtagactatgcattgttattcattataatattttttgctgtcataatcgcctcata

SEQ ID NO: 23 - DNA, 25 bp, Artificial
  Probe
aaccaatttgtgttgttctgattca

SEQ ID NO: 24 - DNA, 25 bp, Artificial
  Probe
ttggtttctgggtggccaattcaga

SEQ ID NO: 25 - DNA, 25 bp, Artificial
  Probe
aacagtctccaggaccatcagtata

SEQ ID NO: 26 - DNA, 25 bp, Artificial
  Probe
gtatactgcatttcatgtgcaccaa

SEQ ID NO: 27 - DNA, 25 bp, Artificial
  Probe
gtcactgatcctgcaaatggacatc

SEQ ID NO: 28 - DNA, 25 bp, Artificial
  Probe
atggacatcattttagcacactagc

SEQ ID NO: 29 - DNA, 25 bp, Artificial
  Probe
agcacactagcggtttatattttaa

SEQ ID NO: 30 - DNA, 25 bp, Artificial
  Probe
tttaaggaccttcattctctgttct

SEQ ID NO: 31 - DNA, 25 bp, Artificial
  Probe
cagttgcaacttacgcttggcatct

SEQ ID NO: 32 - DNA, 25 bp, Artificial
  Probe
gcttggcatcttcagaatgcttttc

SEQ ID NO: 33 - DNA, 25 bp, Artificial
  Probe
tttttgctgtcataatcgcctcata

SEQ ID NO: 34 - DNA, 285 bp, Homo sapiens
gaaatctgggcttggatgtgtgtgtgtgtgtgtgtgtgtgtgtgtgcacatatgcagtgtggtgggagtggggcaacttggggaatatgttacatgtgtgactgtgttggccctggcgaagtaatgtgtcagaaagggtaaatgtgtggacacttgcaagtgctcatggatgaatttatatgtttgagtcatagaaaaagtgtaccctttgatagaagcacattttctttccaaaggcgtggttattaaccagcagaattatagcaggtatacataacttaagtg

SEQ ID NO: 35 - DNA, 25 bp, Artificial
  Probe
gaaatctgggcttggatgtgtgtgt

SEQ ID NO: 36 - DNA, 25 bp, Artificial
  Probe
gtgtgcacatatgcagtgtggtggg

SEQ ID NO: 37 - DNA, 25 bp, Artificial
  Probe
gtgtggtgggagtggggcaacttgg

SEQ ID NO: 38 - DNA, 25 bp, Artificial
  Probe
gagtggggcaacttggggaatatgt

SEQ ID NO: 39 - DNA, 25 bp, Artificial
  Probe
gttacatgtgtgactgtgttggccc

SEQ ID NO: 40 - DNA, 25 bp, Artificial
  Probe
gttggccctggcgaagtaatgtgtc

SEQ ID NO: 41 - DNA, 25 bp, Artificial
  Probe
gtgtggacacttgcaagtgctcatg

SEQ ID NO: 42 - DNA, 25 bp, Artificial
  Probe
acttgcaagtgctcatggatgaatt

SEQ ID NO: 43 - DNA, 25 bp, Artificial
  Probe
agtgtaccctttgatagaagcacat

SEQ ID NO: 44 - DNA, 25 bp, Artificial
  Probe
aaggcgtggttattaaccagcagaa

SEQ ID NO: 45 - DNA, 25 bp, Artificial
  Probe
atagcaggtatacataacttaagtg